(19) United States
(12) Patent Application Publication
(10) Pub. No.: US 2026/0290533 A1
(43) Pub. Date: Sep. 24, 2026

(54) SYSTEM AND METHOD FOR SUBJECT-EFFICIENT CLINICAL RANDOMIZED CONTROLLED TRIALS

(71) Applicant: The Trustees of Princeton University, Princeton, NJ (US)

(72) Inventors: Sayeri LALA, Princeton, NJ (US); Niraj K. JHA, Princeton, NJ (US)

(73) Assignee: The Trustees of Princeton University, Princeton, NJ (US)

(21) Appl. No.: 19/474,002

(22) PCT Filed: Apr. 12, 2024
(86) PCT No.: PCT/US2024/024200

§ 371 Date: Oct. 9, 2025

Related U.S. Application Data
(60) Provisional application No. 63/458,757, filed on Apr. 12, 2023.
(60) Provisional application No. 63/603,999, filed on Nov. 29, 2023.

(51) Int. Cl. G16H 20/10; G16H 50/70

(52) U.S. Cl. CPC G16H 20/10; G16H 50/70

(57) ABSTRACT

Systems and methods for making a randomized control trial (RCT) more data efficient may include receiving subject data for subjects in control arm(s) or treatment arm(s) of an already-conducted RCT; estimating individual treatment effects (ITEs) by utilizing synthetic intervention (SI) to estimate counterfactual treatment responses for each patient in the control arm and counterfactual control responses for each patient in the treatment arm; conducting a two-sided hypothesis test, using the ITE for each patient and the control data; estimating a null distribution of the ITE for each patient under a null hypothesis by repeatedly resampling the control data to provide samples, and running SI with the samples; using the null distribution to determine a critical value achieving a desired significance level; and conducting the two-sided hypothesis test using the critical value. The methods may include conducting a pilot study and trend-adaptive algorithm to arrive at a sample size estimate.

### CROSS-REFERENCE TO RELATED APPLICATIONS

The present application claims priority to U.S. Provisional Patent Application Nos. 63/458,757, filed Apr. 12, 2023, and 63/603,999, filed Nov. 29, 2023. The contents of each are incorporated by reference herein in its entirety.

### STATEMENT REGARDING FEDERALLY SPONSORED RESEARCH OR DEVELOPMENT

This invention was made with government support under Grant No. CNS-1907381 awarded by the National Science Foundation. The government has certain rights in the invention.

### TECHNICAL FIELD

The present disclosure is drawn to techniques for enhancing the effectiveness of clinical trials, such as may be utilized during the pharmaceutical drug development process.

### BACKGROUND

Clinical trials are performed in phases in order to characterize safety and effectiveness of new drugs prior to market approval. In particular, while Phase-1 and Phase-2 trials gather initial evidence on drug effects using small sample sizes, typically less than 100 subjects per arm, in order to identify tolerable and potentially effective dosages, Phase-3 trials establish safety and effectiveness over candidate dosages by using Randomized Controlled Trials (RCTs) with large sample sizes, typically hundreds to thousands of subjects. Despite Phase-3 trials accounting for 60% of R&D investment for clinical trials (approximately $500 million USD per drug in Year 2019), 30-40% of these trials fail to proceed to market approval, primarily because they fail to establish effectiveness; in other words, these studies lack appropriate sample sizes to reach target power and target significance level.

Techniques for making the drug development process more efficient are therefore useful and desirable.

### BRIEF SUMMARY

Various deficiencies in the prior art are addressed below by the disclosed compositions of matter and techniques.

In various aspects, a method for making a randomized control trial (RCT) more data efficient may be provided. The method may include receiving data of a plurality of subjects. The data may include control data and treatment data. Each subject of the plurality of subjects may be in one arm of a plurality of arms of an already-conducted RCT, such as a first arm (which may be a control arm) or a second arm (which may be a corresponding treatment arm) of an already-conducted RCT. The data may be free of data from a cross-over trial and external data.

The method may include estimating a plurality of individual treatment effects (ITEs) by utilizing synthetic intervention (SI) to estimate a counterfactual treatment response for each patient in the first arm and utilizing a counterfactual control response for each patient in the second arm. The plurality of ITEs may include an ITE for each patient in the first arm and the second arm.

The method may include conducting a two-sided hypothesis test, using the ITE for each patient and the control data. Conducting the test may include estimating a null distribution of the ITE for each patient under a null hypothesis by repeatedly resampling the control data to provide samples, and running SI with the samples. Conducting the test may include using the null distribution to determine a critical value achieving a desired significance level. Conducting the test may include conducting the two-sided hypothesis test using the critical value.

The method may include reporting results of the two-sided hypothesis test. The plurality of subjects includes fewer subjects than a standard RCT would require to reach a target power and significance level with respect to a target average treatment effect (ATE).

The method may include, prior to using SI to estimate the counterfactual treatment response, tuning at least one hyperparameter of the SI, by splitting the control data into training and validation sets, initializing a set of best hyperparameters and sweeping over a range of candidate values for ridge regression regularization strength (λᵣᵣ) and singular value truncation threshold (λₛ). The method may include tracking validation performance under each hyperparameter configuration, where the validation performance is a R² score between true post-intervention validation data and corresponding SI-derived predictions. In some embodiments, to obtain the SI-derived predictions per unit, SI is run with candidate hyperparameters, the training data as donor data, and a target unit data, and wherein the SI is configured to estimate a post-intervention trajectory for a target unit by denoising donor data, learning donor weights with ridge regression on a pre-intervention/baseline data, and using the learned donor weights to predict a target unit's post-intervention trajectory from the donor data. This may further include updating the set of best hyperparameters if the validation performance improves.

The method may include conducting a pilot study to provide initial estimates on parameters defining a standard sample size formula for a one-sample two-sided test. The method may include implementing a trend-adaptive algorithm configured to iteratively accrue RCT data, initialized from the pilot study. The method may include terminating the trend-adaptive algorithm if it is determined to be futile.

In various aspects, a non-transitory computer-readable storage device may be provided. The storage device may contain instructions that, when executed by at least one processing units, cause the processing unit(s) to (collectively) perform an embodiment of the method as disclosed herein.

In various aspects, a system may be provided. The system may include one or more processors. The system may include a memory coupled to the one or more processing units. The system may include a non-transitory computer-readable storage device containing instructions that, when executed by the processing unit(s), may cause the processing unit(s) to (collectively) perform an embodiment of the method as disclosed herein.

### BRIEF DESCRIPTION OF DRAWINGS

The accompanying drawings, which are incorporated in and constitute a part of this specification, illustrate embodiments of the present invention and, together with a general description of the invention given above, and the detailed description of the embodiments given below, serve to explain the principles of the present invention.

FIG. 1 is a schematic illustration of a system.

FIG. 2 is a flowchart of a Trend-Adaptive Design with a Synthetic-Intervention-Based Estimator (“TAD-SIE”).

FIG. 3 is a flowchart of a “conduct_pilot_study” process.

FIG. 4 is a flowchart of a “get_ATE” process.

FIG. 5 is a flowchart of a “get_variance_of_ATE” process.

FIG. 6 is a flowchart of a “run_sample_size_search” process.

FIG. 7 is an algorithm for a “get_step_size” process.

FIG. 8 is an algorithm for a “check_for_futility” process.

FIG. 9 is a flowchart of Subject-Efficient Clinical Randomized Controlled Trials using Synthetic Intervention (“SECRETS”) process.

FIG. 10 is a flowchart of a “estimate_ITEs” process.

FIG. 11 is an algorithm for a “tune_SI_hyperparams” process.

FIG. 12 is an algorithm for an “SI” process.

FIG. 13 is an algorithm for a “run_hypothesis_test” process.

FIG. 14 is an algorithm for a “sample_null” process.

FIG. 15 is an algorithm for a “tune_critical_value” process (sometimes referred to as “get_cv”).

FIGS. 16A and 16B are graphs showing the effect of futility_power_boundary and step_size_scale_factor on the significance level (16A) and power (16B) under TAD-SIE with 1−β_{target}=80% and α_{target}=5%.

FIG. 17 is a graph showing conditional power under two-sided testing as a function of z, the interim test statistic, and t, the information fraction (formula specified in line 2 of Algorithm 2); the function is plotted over nonnegative values of z since the function is symmetric in z.

FIG. 18 is a graph showing significance level and power under different values of futility_power_boundary and step_size_scale_factor for TAD-SIE with 1−β_{target}=80% and α_{target}=5%. Each point corresponds to a different value of step size scale factor that has been sampled over the following set of values: {0.1, 0.2, 0.3, 0.4, 0.5, 0.6}.

FIG. 19 is a graph showing Significance level and power under different values of futility_power_boundary and step_size_scale_factor for TAD-SIE with 1−β_{target}=90% and α_{target}=5%. Each point corresponds to a different value of step size scale factor that has been sampled over the following set of values: {0.1, 0.2, 0.3, 0.4, 0.5, 0.6}.

FIGS. 20A and 20B are box plots over final arm size (20A) and number of iterations (20B) for TAD-SIE with 1−β_{target}=80% and α_{target}=5% across different values of step_size_scale_factor when either H₁ or H₀ holds.

FIGS. 21A and 21B are box plots over final arm size (21A) and number of iterations (21B) for TAD-SIE with 1−β_{target}=90% and α_{target}=5% across different values of step_size_scale_factor when either H₁ or H₀ holds.

FIGS. 22A and 22B are box plots over final arm size (22A) and number of iterations (22B) when either H₁ or H₀ holds for the sample-efficient and time-efficient modes of TAD-SIE with 1−β_{target}=80% and α_{target}=5%.

FIGS. 23A and 23B are box plots over final arm size (23A) and number of iterations (23B) when either H₁ or H₀ holds for the sample-efficient and time-efficient modes of TAD-SIE with 1−β_{target}=90% and α_{target}=5%.

FIGS. 24A and 24B are box plots over final arm size (24A) and number of iterations (24B) under the sample-efficient mode of TAD-SIE using the naive approach or a disclosed exemplary approach for estimating moments with 1−β_{target}=80% and α_{target}=5%.

FIGS. 25A and 25B are box plots over final arm size (25A) and number of iterations (25B) under the time-efficient mode of TAD-SIE using the naive approach or a disclosed exemplary approach for estimating moments with 1−β_{target}=80% and α_{target}=5%.

FIGS. 26A and 26B are graphs showing the effect of moment estimation strategy used for calculating CP (e.g., naïve vs the disclosed exemplary approach) on significance level (26A) and power (26B) for the sample-efficient mode of TAD-SIE with 1−β_{target}=80% and α_{target}=5%.

FIGS. 27A and 27B are graphs showing the effect of moment estimation strategy used for calculating CP (e.g., naïve vs the disclosed exemplary approach) on significance level (26A) and power (26B) for the time-efficient mode of TAD-SIE with 1−β_{target}=80% and α_{target}=5%.

It should be understood that the appended drawings are not necessarily to scale, presenting a somewhat simplified representation of various features illustrative of the basic principles of the invention. The specific design features of the sequence of operations as disclosed herein, including, for example, specific dimensions, orientations, locations, and shapes of various illustrated components, will be determined in part by the particular intended application and use environment. Certain features of the illustrated embodiments have been enlarged or distorted relative to others to facilitate visualization and clear understanding. In particular, thin features may be thickened, for example, for clarity or illustration.

### DETAILED DESCRIPTION

The following description and drawings merely illustrate the principles of the invention. It will thus be appreciated that those skilled in the art will be able to devise various arrangements that, although not explicitly described or shown herein, embody the principles of the invention and are included within its scope. Furthermore, all examples recited herein are principally intended expressly to be only for illustrative purposes to aid the reader in understanding the principles of the invention and the concepts contributed by the inventor(s) to furthering the art and are to be construed as being without limitation to such specifically recited examples and conditions. Additionally, the term, “or,” as used herein, refers to a non-exclusive or, unless otherwise indicated (e.g., “or else” or “or in the alternative”). Also, the various embodiments described herein are not necessarily mutually exclusive, as some embodiments can be combined with one or more other embodiments to form new embodiments.

The numerous innovative teachings of the present application will be described with particular reference to the presently preferred exemplary embodiments. However, it should be understood that this class of embodiments provides only a few examples of the many advantageous uses of the innovative teachings herein. In general, statements made in the specification of the present application do not necessarily limit any of the various claimed inventions. Moreover, some statements may apply to some inventive features but not to others. Those skilled in the art and informed by the teachings herein will realize that the invention is also applicable to various other technical areas or embodiments. Disclosed is a novel framework that solves the problem of sample size estimation for clinical parallel-group RCTs by using a new trend-adaptive algorithm tailored to a powerful hypothesis testing scheme (sometimes referred to herein as Subject-Efficient Clinical Randomized Controlled Trials using Synthetic Intervention or “SECRETS”).

To ensure that any Phase-3 trial succeeds and thereby make the drug development process more efficient, a new solution called Trend-Adaptive Design with a Synthetic-Intervention-Based Estimator (TAD-SIE) is provided that implements a parallel-group RCT, a standard design, of adequate sample size in order to reach target power and target significance level. To do this, TAD-SIE adopts SECRETS, a state-of-the-art hypothesis testing scheme, which simulates a cross-over trial in order to increase power over standard hypothesis testing. Using SECRETS increases the chance of reaching target power within a trial's sample size constraints, especially under noisy estimates of the average treatment effect (ATE) and variance of the primary outcome, i.e., the outcome defining the ATE. Then, to estimate the sample size required for SECRETS, TAD-SIE implements a novel trend-adaptive design (TAD) that gradually increases the sample size while refining estimates of the ATE and outcome variance under SECRETS based on accumulated RCT data in order to converge to a sample size that yields the target power. Meanwhile, the algorithm controls significance level by incorporating futility stopping. Given its iterative nature, TAD-SIE can reach the target operating point by trading off final sample size with number of iterations. Therefore, also disclosed are sample-efficient and time-efficient modes for TAD-SIE that can be chosen based on trial constraints.

Trial designers calculate the sample size required for an RCT by using the hypothesis testing framework. For example, for a two-arm, parallel group, superiority trial with an equal number of participants per arm, a common type of RCT design, the sample size formula is determined by a two-sample, two-sided hypothesis test over the value of the ATE δₜᵣᵤₑ, where the null hypothesis (H₀) posits that δₜᵣᵤₑ=0 and the alternative hypothesis (H₁) posits δₜᵣᵤₑ=8, where δ>0. Under this setup, the sample size required to obtain a power of 1−β_{target} (%) and significance level of α_{target} (%) is given by Eq. (1), where n_{α} is the arm size, σ_{control}² and σₜᵣₑₐₜ² are the variances of the primary outcome under the control and treatment groups, respectively, and z_{1-α}_{_{target}}_{/2} and z_{1-β}_{_{target}} correspond to the (1-α_{target}/2)th and (1−β_{target})th percentiles of a standard normal distribution.

$${n}_{a} = \frac{( {σ}_{control}^{2} + {σ}_{t r e a t}^{2} ) {( {z}_{1 - {α}_{t a r g e t}} + {z}_{1 - β} )}^{2}}{{δ}^{2}} \qquad ( 1 )$$

Trial designers set the target power and target significance level, with power typically above 80% and significance level below 5%, and can also set δ or the standardized treatment effect (i.e., the ratio of ATE to standard deviation) to the smallest clinically meaningful value that is of interest to detect in the absence of any estimates of these parameters [9]. Otherwise, δ, σ_{control}² and σₜᵣₑₐₜ² can be estimated from prior studies (e.g., RCTs or external pilot studies) if available or internal pilot studies conducted by trial designers; however, calculations based on these data sources, especially pilot studies, tend to overestimate treatment effects and underestimate variances since the data fail to capture the distribution of the ATE over the target population, thereby resulting in underpowered studies.

Adaptive trial designs have been proposed as a way to calculate appropriate sample sizes, given noisy initial sample size estimates. Specifically, adaptive designs adjust initial estimates based on interim analyses conducted over the course of the trial (up till reaching the initial planned sample size), where at each interim analysis, the outcome data from participants entered thus far into the trial are reviewed. Standard approaches for doing so include Group Sequential Designs (GSD), Stochastic Curtailment, and TADs.

A GSD can decrease the initial planned sample size through repeated testing, i.e., testing at each interim analysis. Specifically, a GSD sequentially acquires RCT data from groups of subjects at each interim analysis and performs statistical testing at each analysis based on all the RCT data acquired thus far. In order to control the significance level, the test boundary used at each analysis, especially any preceding the final one, needs to be increased relative to the test boundary used by a nonadaptive trial; for example, for a two-sided test with 5% significance level, a GSD with two planned analyses would use a critical value of 2.78 at the first analysis and 1.97 at the second analysis under an O'Brien-Fleming model, compared to a conventional value of 1.96 used by a nonadaptive design. Consequently, a GSD yields marginal reductions in sample sizes when the standardized treatment effect is larger than that used for planning [12]. For GSDs to be useful, a trial designer still needs to have a good prior over the range of the standardized treatment effect, otherwise risks underpowering or overpowering the study [12], where the latter consequence is not ideal since it consumes substantially more resources than necessary.

Stochastic Curtailment is another approach that can decrease the initial planned sample size by terminating trials that appear futile, that is, likely to fail in reaching statistical significance. It determines futility based on Conditional Power (CP), which extrapolates power at the final sample size (i.e., the initial estimate of the sample size) conditioned on the value of the interim test statistic. For example, for a one-sided hypothesis test with significance level α, CP is determined by Eq. (2), where t is the information fraction, defined as the ratio of the sample size at interim analysis to the planned sample size, z is the test statistic calculated from interim data, Z is the random variable corresponding to the test statistic at the final sample size, zₐ is the test boundary given by the αth percentile under the standard normal distribution, δ is the ATE to test against under H₁, and Φ is the cumulative distribution function for the standard normal distribution. If CP at any interim analysis lies below some pre-specified futility threshold, the trial is terminated. While Stochastic Curtailment can be used to control the significance level and reduce resources expended by terminating early, it reduces power and therefore cannot be used to appropriately power studies.

$$C P ( t , z ) = P r ( Z > {z}_{α} \vert t , z , δ ) = 1 - Φ [ \frac{{z}_{α} - z \sqrt{t} - δ ( 1 - t )}{\sqrt{1 - t}} ] \qquad ( 2 )$$

Instead of decreasing the initial planned sample size, TADs can increase it based on trends observed from interim data. Among trend-adaptive algorithms, those based on CP have been recommended since they can control significance level without making statistical adjustments to the test statistic and test critical value. They do this by only permitting sample size increases when the trend in the data appears “promising,” a condition determined by CP at interim analysis. In particular, CP is evaluated based on the data at information fraction t, i.e., δ=\hat{δ}=z/\sqrt{t}, which simplifies Eq. (2) to Eq. (3), where the second line follows from the relationship that 1-Φ(x)=Φ(−x). If CP is sufficiently high, i.e., 50%, or lies within a promising range, where the lower bound is determined by the sample size at interim analysis, the initial planned sample size, the maximum sample size, and target power, and the upper bound is given by target power, the final sample size can be increased according to the sample size formula based on revised parameter estimates obtained from interim data or according to CP by setting CP to the target power and solving it for sample size, given the revised parameter estimates. In practice, such TADs have marginal impact on increasing power since the probability of satisfying the CP criterion at an interim analysis remains low.

$$C P ( t , z ) = P r ( Z > {z}_{α} \vert t , z , \hat{δ} ) = 1 - Φ [ \frac{{z}_{α}}{\sqrt{1 - t}} - \frac{z}{\sqrt{t ( 1 - t )}} ] = Φ [ \frac{z}{\sqrt{t ( 1 - t )}} - \frac{{z}_{α}}{\sqrt{1 - t}} ] \qquad ( 2 )$$

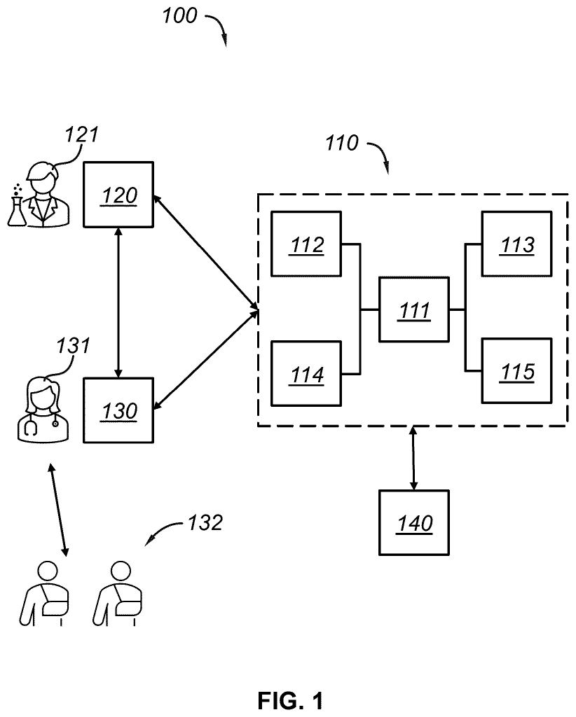

Referring to FIG. 1, a system (100) for designing, and optionally performing, clinical trials may be provided. The system (100) may include a first system (110) for making a randomized control trial (RCT) more data efficient. The first system (110) may include one or more processing units (111) operably coupled to a memory (112), a non-transitory computer-readable storage device (113), a communications interface (114), and one or more input/output devices (115) (e.g., a display, a mouse, a keyboard, etc.) The processing unit(s) may be operably coupled to additional components as needed to perform the various tasks.

As used herein, the term “processing unit” may refer to any CPU, GPU, core, hardware thread, or other processing construct. As used herein, the term “thread” refers to any software or processing unit or arrangement thereof that is configured to support the concurrent execution of multiple operations.

The non-transitory computer-readable storage device may contain instructions that, when executed by at least one processing unit, may cause the processing unit(s) to, collectively, perform steps of a method for making a randomized control trial (RCT) more data efficient as disclosed herein.

The first system (110) may be configured to operably communicate with one or more first remote processing units (120) that may be used by one or more researchers (121), scientists, or other users who would like to design an appropriate RCT.

The first system (110) may be configured to operably communicate with one or more second remote processing units (130) that may be used by one or more researchers (131), scientists, clinicians, etc., who would are or intend to conduct an appropriate RCT involving a plurality of subjects (132), which are preferably human subjects, who may have one or more conditions. The second remote processing unit(s) may be configured to send communications to the system. The second remote processing unit(s) may be configured to second communications to the first processing unit(s). This may include, e.g., test data or data related to the subjects.

The first system (110) may be configured to operably communicate with one or more third remote processing units (140). The third remote processing units may include, e.g., one or more databases or applications accessible via one or more application programming interfaces (APIs).

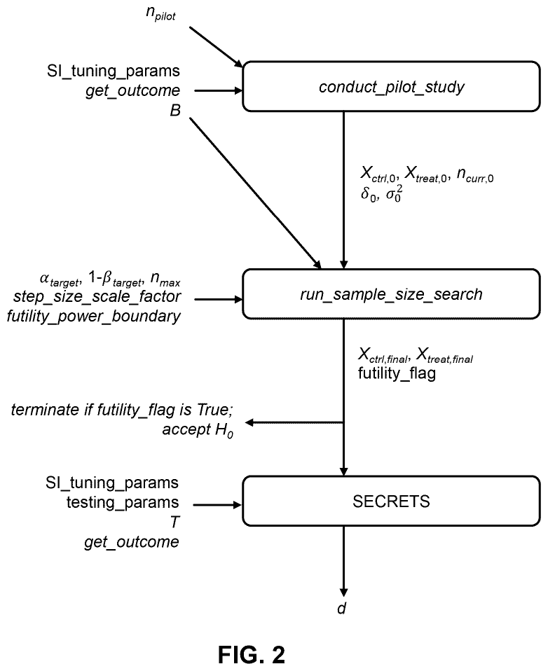

The method may include various steps. As seen in FIG. 2, a flowchart of an embodiment of TAD-SIE is shown.

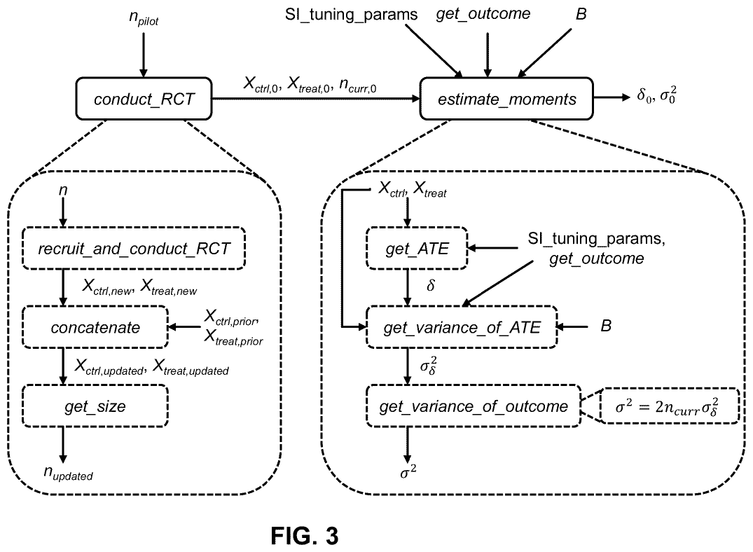

As seen, TAD-SIE may first call conduct_pilot_study (see FIG. 3), which runs an internal pilot study to obtain initial estimates for these parameters under SECRETS (discussed in more detail below). Specifically, it takes in as input nₚᵢₗₒₜ, the arm size for the control/treatment group under the pilot study, SI_tuning_params, a dictionary containing hyperparameters for SI, and get_outcome, a function calculating the outcome metric from a patient's response trajectory, both of which are used for estimating ITEs, as done in SECRETS, and B, a hyperparameter used for estimating the variance of the ITEs. conduct_pilot_study outputs the initial RCT data for the control and treatment groups, X_{ctrl,0} and X_{treat,0}, respectively, the arm size of the initial control and treatment groups, n_{curr,0}, along with initial estimates of the ATE and variance of the ITE, δ₀ and σ₀², respectively.

TAD-SIE then calls run_sample_size_search to iteratively refine the estimates to converge to an accurate estimate of the target sample size needed for target power while stopping for futility to control the significance level. To do this, it uses the RCT data collected from the pilot study, the initial estimates of the ATE and variance, along with hyperparameters, which include α_{target}, the target significance level, 1−β_{target}, the target power, nₘₐₓ, the maximum arm size allowed by the trial, step_size_scale_factor, the rate at which the sample size is increased, and futility_power_boundary, the threshold that determines if futility holds. run_sample_size_search outputs the complete RCT data collected over the trial, X_{ctrl,final} and X_{treat,final}, and futility flag, which indicates whether the trial is futile.

If the trial is futile, TAD-SIE fails to reject the null hypothesis and accepts it by convention. Otherwise, TAD-SIE runs SECRETS to perform hypothesis testing and return the test outcome d given the complete RCT data, using the hyperparameters given by SI_tuning_params, testing_params, and T, and function get_outcome.

Next, we describe each step of TAD-SIE in more detail.

The method generally begins by initializing a search with a pilot study. See FIG. 3. conduct_pilot_study provides initial estimates on parameters defining the standard sample size formula for a one-sample two-sided test (e.g., the testing scheme used by SECRETS), which is given by Eq. (4).

$$n = \frac{{{σ}^{2} ( {z}_{1 - α / 2} + {z}_{1 - β} )}^{2}}{{δ}^{2}} \qquad ( 4 )$$

Specifically, it estimates the moments, i.e., the ATE, δ, and variance of the ITE, σ², under SECRETS.

Referring to FIG. 5, to estimate δ and σ², conduct_pilot_study runs an internal pilot study by collecting RCT data over a small arm size of nₚᵢₗₒₜ with conduct_RCT. conduct_RCT calls recruit and conduct RCT to run an RCT at the given arm size by recruiting subjects, randomizing them over control and treatment groups, and monitoring their responses up through the RCT endpoint. It then calls concatenate to merge the newly collected RCT data over the control and treat arms, X_{ctri,new} and X_{treat,new}, respectively, with any prior collected data, X_{ctrl,prior} and X_{treat,prior} (there is no prior data for the pilot study). It also calculates the current arm size of the RCT with get size and returns the updated data and arm size.

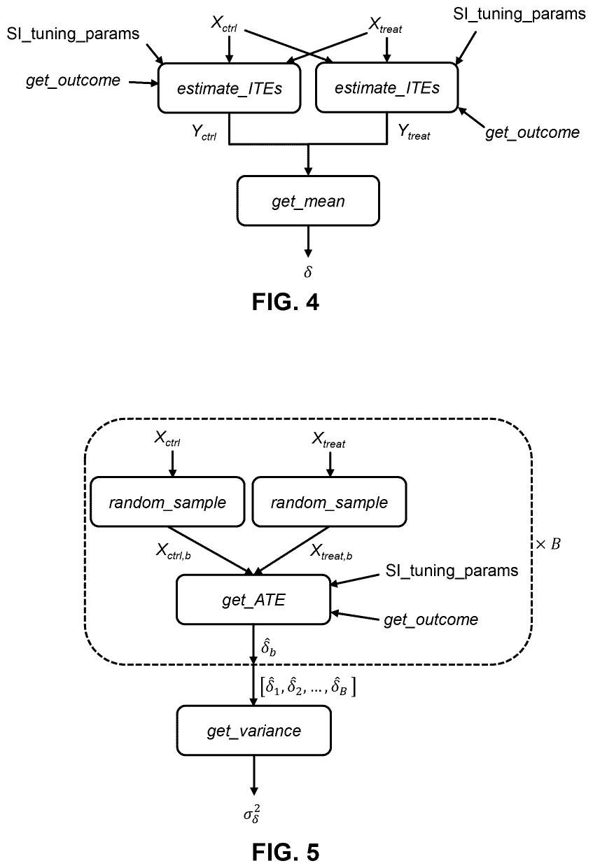

Afterwards, conduct_pilot_study estimates the moments under SECRETS from the RCT data collected from the pilot study, X_{ctrl,0} and X_{treat,0}, using estimate moments, which uses SI_tuning_params, get_outcome, and hyperparameter B. estimate moments first estimates δ₀ by calling get_ATE, illustrated in FIG. 4. get_ATE first calculates the ITEs, Y_{ctrl} and Yₜᵣₑₐₜ, for the control and treatment groups, respectively, using estimate ITEs with the collected RCT data, X_{ctrl} and Xₜᵣₑₐₜ, hyperparameters defined by SI_tuning_params, and function get_outcome, and then averages the ITEs with get mean.

Then, estimate_moments calculates σ₀² but uses a new procedure to do so because the sample size formula given by Eq. (4) requires that the ITEs be independently and identically distributed (i.i.d.) according to a Gaussian, although the independence assumption is violated under SECRETS because SI induces dependencies among the ITEs. Given that the distribution of the ATE under SECRETS is approximated by a normal distribution, per the theorem on Dependency Neighborhoods based on Stein's method, estimate moments first estimates σ_{δ}², the variance of the ATE, and uses it to estimate σ², the variance of a set of hypothetical ITEs satisfying the i.i.d. assumption that would also yield the observed distribution of the ATE.

To estimate σ_{δ}², estimate moments calls get_variance_of_ATE, illustrated in FIG. 5. get_variance_of_ATE estimates the variance of the ATE in a fashion similar to the sample_null procedure used in SECRETS (described below) for estimating the variance of the ATE under the null distribution. Specifically, get_variance_of_ATE first samples from the distribution of the ATE, where at each sampling iteration b, get_variance_of_ATE draws one bootstrap sample from the RCT data over the control arm, X_{ctrl,b}, and one bootstrap sample from the RCT data over the treatment arm, X_{treat,b}; this is different from sample null, which samples from the control arm only to produce samples from the null distribution. Afterwards, get_variance_of_ATE calls get_ATE to calculate \hat{δ}_{b}, the ATE associated with the bootstrap samples. After sampling B times from the distribution of the ATE, the variance of the ATE, i.e., σ_{δ}², is given by the variance over the B samples. Afterwards, estimate moments calls get_variance_of_outcome, which calculates σ² according to the relationship between variance of a mean and variance of underlying i.i.d. samples, given by Eq. (5), where X̅ is the mean over a set of n i.i.d samples, {X₁, X₂, . . . , Xₙ}.

$$\begin{matrix}{σ}_{\overline{X}}^{2} := Var ( \overline{X} ) \\ := Var ( \frac{{\sum }_{i = 1}^{n} {X}_{i}}{n} ) \\ = \frac{Var ( {X}_{i} )}{n} \\ := \frac{{σ}^{2}}{n}\end{matrix} \qquad ( 5 )$$

Specifically, σ² is calculated by multiplying σ_{δ}² by 2n_{curr} since the ITEs are pooled over the two arms, where n_{curr} is the arm size of the control/treatment group.

The method may then include Sample Size Estimation using a Trend-Adaptive Design. The sample size formula works well when δ and σ² are estimated correctly. Hence, evaluations based on estimates of δ and σ² from small samples (e.g., as with the pilot study) are expected to extrapolate poorly. In addition, under TAD-SIE's procedure for estimating moments, the formula's extrapolation capacity further diminishes because the variance calculated by estimate moments can scale with the sample size n, i.e., σ²=O(n).

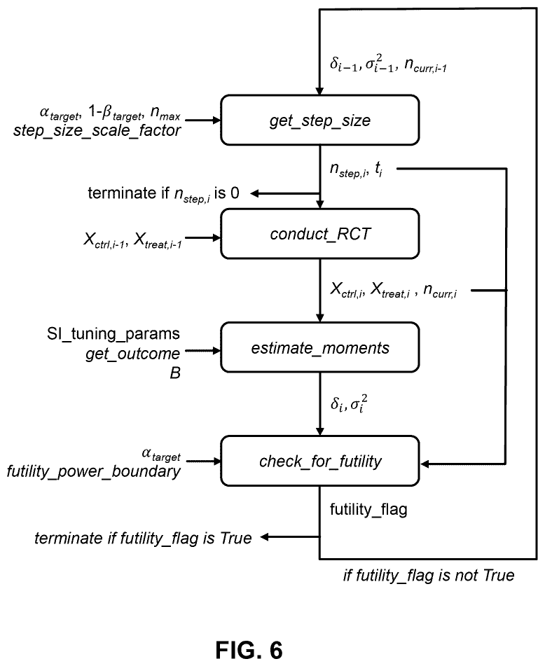

Therefore, TAD-SIE implements a trend-adaptive algorithm tailored to SECRETS, called run_sample_size_search, which iteratively accrues the RCT data, initialized from the pilot study, till a sufficient number of samples has been collected to reach target power (subject to resource constraints) or the trial terminates for futility. The flowchart of run sample size search is illustrated in FIG. 6.

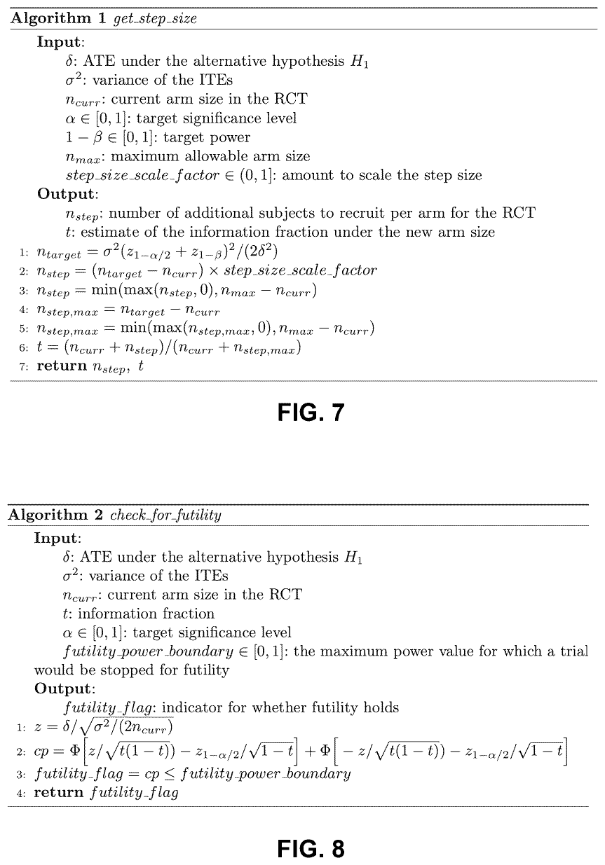

At iteration i, run_sample_size_search first increases the current sample size according to get_step_size, which determines the step size n_{step,i} and information fraction tᵢ based on quantities calculated from the prior iteration (or the pilot study if i=0), i.e., the moments δᵢ₋₁, σᵢ₌₁², and
arm size n_{curr,i-1}, and hyperparameters set by the trial designer, i.e., α_{target}, 1−β_{target}, nₘₐₓ, and
step_size_scale_factor. The pseudocode for get step size is given in Algorithm 1 shown in FIG. 7. Specifically, get_step_size first calculates n_{target}, an estimate of the arm size needed for target performance, using the sample size formula (Eq. (4)) evaluated with target significance level α, target power 1−β, and moments δ and σ²; it divides by two since the total sample size is split between the two arms (line 1). Then, get step size calculates the step size nₛₜₑₚ by calculating the difference between the target arm size n_{target} and current arm size n_{curr} and scaling it according to step_size_scale_factor, which is less than 1 to guard against overpowering the study (line 2). The step size is constrained so that it is nonnegative (decreases are not allowed since the data have already been collected) and does not exceed the maximum allowable step size set by the maximum arm size nₘₐₓ (line 3). In addition, get_step_size estimates the information fraction t resulting from the sample size increase, which is used for futility stopping. t is obtained by calculating the maximum possible step size given by n_{step,max} (lines 4-5) and taking the ratio between the updated arm size and the maximum possible value for the updated arm size, the latter quantity serving as an estimate of the final target arm size (line 6).

If the step size is zero, run_sample_size_search terminates. Otherwise, it collects RCT data over n_{step,i} additional subjects per arm using conduct RCT, which also takes in the RCT data collected at the prior iteration, i.e., X_{ctrl,i-1} and X_{treat,i-1}, to yield the accumulated RCT data over the control and treatment arms, X_{ctrl,i} and X_{treat,i}, respectively, and the updated arm, size n_{curr,i}.

Afterwards, run_sample_size_search calls estimate moments with the updated RCT data, X_{ctrl,i} and X_{treat,i}, hyperparameters, i.e., SI_tuning_params and B, and function get_outcome to yield updated estimates of the moments, given by δᵢ and σᵢ².

Finally, run_sample_size_search checks for futility using check_for_futility, outlined in Algorithm 2 shown in FIG. 8, which uses quantities calculated in the current iteration of run_sample_size_search, i.e., the moments δᵢ and σᵢ², the current arm size n_{curr,i}, and information fraction tᵢ, along with hyperparameters α_{target} and futility_power_boundary. Specifically, check_for_futility assesses futility with Conditional Power (CP), following Stochastic Curtailment. First, check for futility calculates the interim test statistic z using δ, σ², and the current arm size, n_{curr}; the variance of the ATE is calculated using Eq. (5), where the total number of samples is 2n_{curr} since the ITEs are pooled over the two arms (line 1). Then it evaluates CP at information fraction t, interim test statistic z, and target significance level α using the formula adapted for two-sided testing (line 2) and marks the trial as futile if the estimate of CP is below futility power boundary (line 3). run_sample_size_search terminates if the trial is deemed futile; otherwise, it continues to the next iteration. The algorithm eventually terminates by futility or by reaching the maximum arm size nₘₐₓ. Setting step_size_scale_factor determines how fast the algorithm terminates, with larger values resulting in fewer iterations at the cost of larger sample sizes since the sample size is increased at a higher rate. Given this tradeoff, the trial designer can implement more of a sample-efficient mode (TAD-SIE-SE) or more of a time-efficient mode (TAD-SIE-TE) by setting step_size_scale_factor to a relatively low or high value, respectively. It will be understood that this there is no fixed value that delineates sample-efficient from time-efficient mode. Rather, increasing the value of step_size_scale_factor will gradually change the mode from being more sample efficient to more time-efficient (or the reverse, if the value is decreased). In some cases, TAD-SIE-SE mode may utilize step_size_scale_factor values of below about 0.35, while TAD-SIE-TE mode may utilize step_size_scale_factor values of about 0.35 or above. In some embodiments, the value of step_size_scale_factor may be capped. In a preferred embodiment, the step_size_scale_factor value may be capped at 0.7 to control futility stopping rates and yield feasible solutions.

After determining the sample size, TAD-SIE performs hypothesis testing. If run_sample_size_search marks the trial as futile, TAD-SIE fails to reject the null hypothesis, in which case it accepts it by convention. Otherwise, TAD-SIE performs hypothesis testing with SECRETS using the final RCT data given by X_{ctrl,final} and X_{treat,final}, along with hyperparameters contained in SI_tuning_params and testing_params, hyperparameter T, and function get_outcome, and returns the test outcome d.

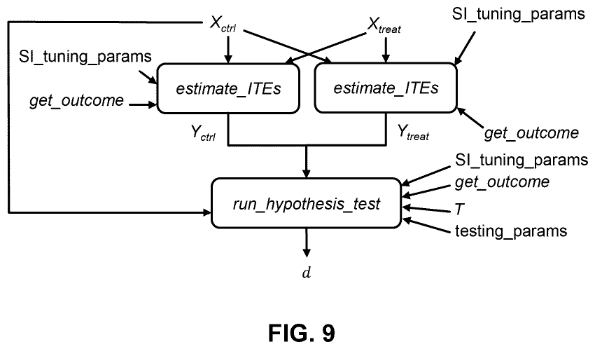

SECRETS, illustrated in FIG. 9, is a hypothesis testing algorithm that increases power of an already-conducted parallel-group RCT by simulating the cross-over design with a state-of-the-art counterfactual estimation algorithm called Synthetic Intervention (SI). The framework for a two-arm superiority trial with an equal number of participants per arm is described herein, as this this a common setup. However, alternative arrangements are envisioned.

First, SECRETS estimates individual treatment effects (ITEs) across all patients in the control and treatment groups, given by Y_{ctrl} and Yₜᵣₑₐₜ, respectively, using the observed patient data, X_{ctrl} and Xₜᵣₑₐₜ, and procedure estimate_ITEs, which takes in hyperparameters for SI, contained in the SI_tuning_params dictionary, and function get_outcome, which calculates the primary outcome of the trial given a patient's response trajectory under some intervention. Then, it performs hypothesis testing using the merged set of ITEs with run_hypothesis_test, which implements a data-driven hypothesis test procedure given that the ITEs calculated underestimate ITEs do not satisfy an assumption under standard hypothesis testing. To implement the new test procedure, run_hypothesis_test uses hyperparameters contained in SI_tuning_params and testing_params, hyperparameter T, and function get_outcome.

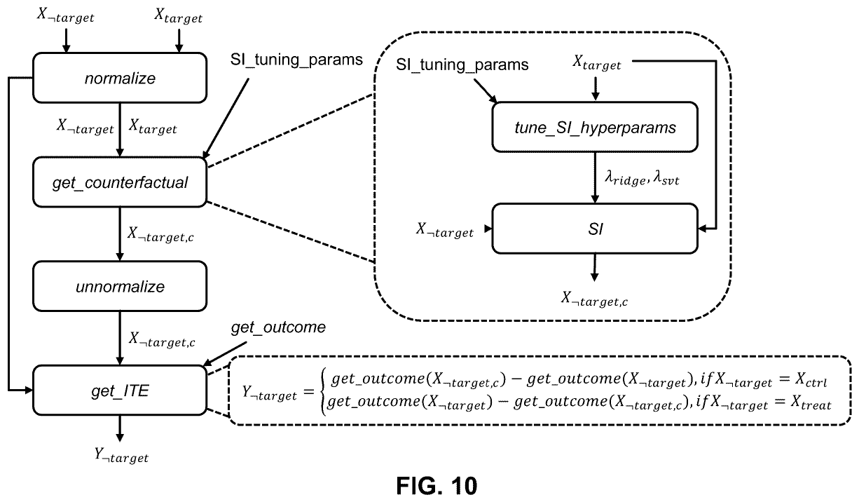

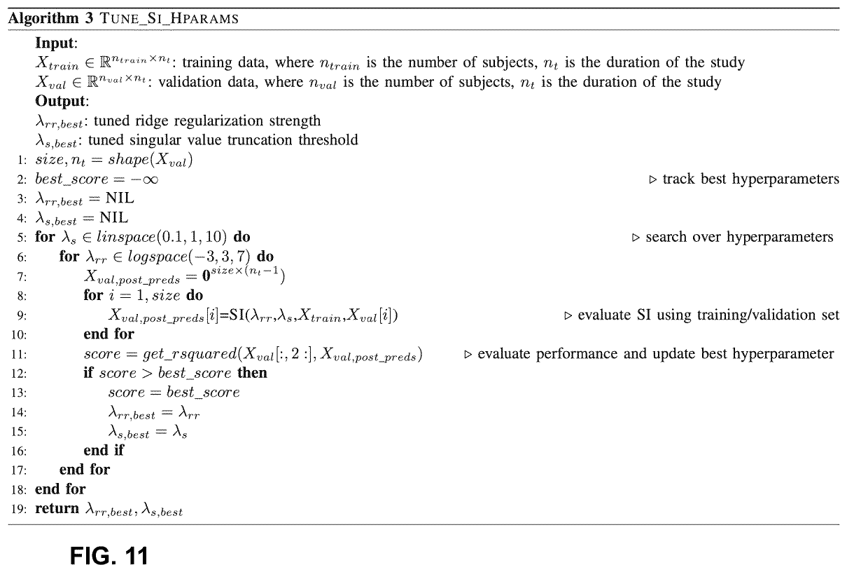

The flowchart of estimate_ITEs is shown in FIG. 10. To estimate an ITE per patient, estimate_ITEs calculates the counterfactual outcome under the target intervention to which the patient was not exposed and then takes the difference between the counterfactual outcome under the target intervention and the observed outcome under the intervention to which the patient was exposed. To do this, estimate_ITEs takes in X_{¬target}, the data of the group unexposed to the target intervention, and X_{target}, the data of the group exposed to the target intervention, normalizes the data, and calls get_counterfactual to calculate X_{¬target,c}, the counterfactual outcome under the target intervention per subject from the unexposed group. get_counterfactual uses SI to do this, which calculates the counterfactual outcome under the target intervention for some target unit using observed outcomes from a set of donor units exposed to the target intervention. Specifically, get_counterfactual tunes SI's regularization hyperparameters with tune_SI_hyperparams, which takes in X_{target} and SI_tuning_params (this contains r_{train,val}, a hyperparameter for the ratio of the training to validation split), and runs SI with the tuned hyperparameters and X_{target} as the donor data and each unit from X_{¬target} as target unit data. tune_SI_hyperparams, seen in Algorithm 3 (FIG. 11) first initializes the set of best parameters (lines 2-4) and then sweeps over a wide range of candidate values for singular value truncation threshold (λₛ) and ridge regression regularization strength (λᵣᵣ), tracking the validation performance under each hyperparameter configuration (lines 5-18), where the validation performance is the R² score between the true post-intervention validation data and corresponding SI-derived predictions. To obtain the SI-derived predictions per unit, tune_SI_hyperparams runs SI with the given candidate hyperparameters and Xₜᵣₐᵢₙ as the donor data, and Xᵥₐₗ[i] as the target unit data (lines 8-10).

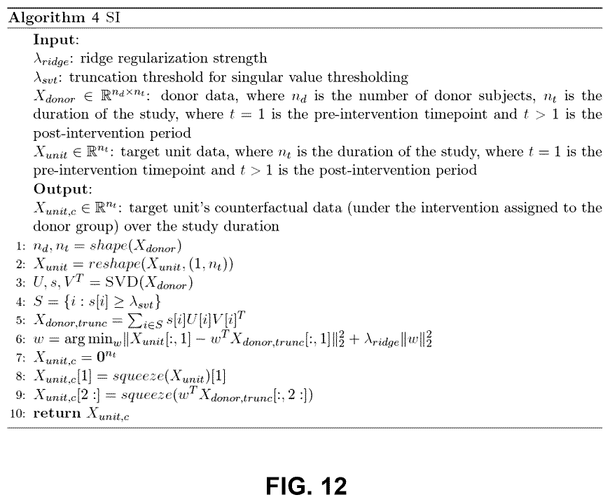

Specifically, SI, shown in Algorithm 4 (FIG. 12) estimates the post-intervention trajectory for the target unit by denoising the donor data (lines 3-5), learning the donor weights with ridge regression on the pre-intervention (i.e., baseline) data (line 6), and using the learned donor weights to estimate the target unit's post-intervention trajectory from the donors' post-intervention data (line 9). Since SI is used to predict the post-intervention trajectory of the target unit, the unit's “counterfactual” pre-intervention data is initialized to its original pre-intervention data (line 8).

After estimating the post-intervention trajectories across the validation units, tune_SI_hyperparams calculates the validation performance; if the validation performance improves, the best hyperparameter configuration is updated (lines 11-16).

After unnormalizing the observed and counterfactual outcomes, estimate_ITEs calls get_ITE to calculate the ITE using X_{¬target}, X_{¬target,c}, and get_outcome. estimate_ITEs is used to calculate Y_{ctrl} by setting X_{¬target} to X_{ctrl} and X_{target} to Xₜᵣₑₐₜ and vice versa to calculate Yₜᵣₑₐₜ.

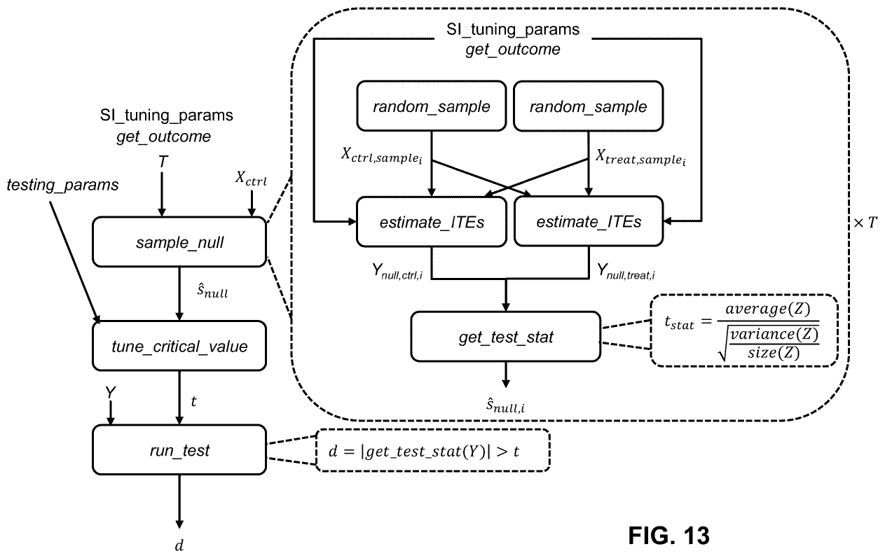

By using the same donor data to estimate the ITE per patient in the same treatment arm, SI induces dependencies among the ITEs, thereby violating the independence assumption under standard hypothesis testing. Therefore, run hypothesis test implements a new test, which uses the same test statistic used by the one-sample t-test but tunes the critical value of the test to control the significance level. The flowchart of run hypothesis test is shown in FIG. 13.

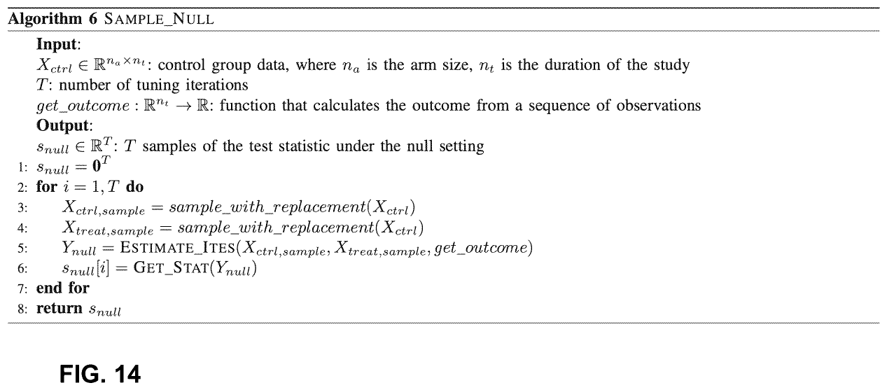

First, the method may call sample null, which estimates the variance of the null distribution of the test statistic using a novel bootstrapping algorithm. sample_null, as shown in Algorithm 6 (FIG. 14). In FIG. 14, samples from the null distribution T times by drawing bootstrap samples from the original control arm data, X_{ctrl}, to construct comparable control and treatment groups, X_{ctrl,sample}_{ᵢ} and X_{treat,sample}_{ᵢ} (lines 3-4). Thus, it calculates their corresponding ITEs using estimate_ITEs (with SI_tuning_params and get_outcome) (line 5). FIG. 14 shows estimate_ites as relying on default hyperparameters; however, in some embodiments, estimate_ites may instead rely on corresponding values stored in SI_tuning_params.

The algorithm then uses the ITEs to calculate the test statistic with get_test_stat. Given samples of the test statistic from the null distribution ŝₙᵤₗₗ, run_hypothesis_test calls tune_critical_value, which tunes the testing critical value to control the significance level using an algorithm similar to binary search, guided by a set of parameters defined under testing params, which include α_{target}, the target significance level, t_{lower}, the lower bound on the critical value search range, tᵤₚₚₑᵣ, the upper bound on the critical value search range, t_{limit,exp}, the amount to shrink or expand the critical value search range, nₛ, the number of candidate critical values over which to search, and δ_{α}_{_{target}}, the error tolerance for the significance level.

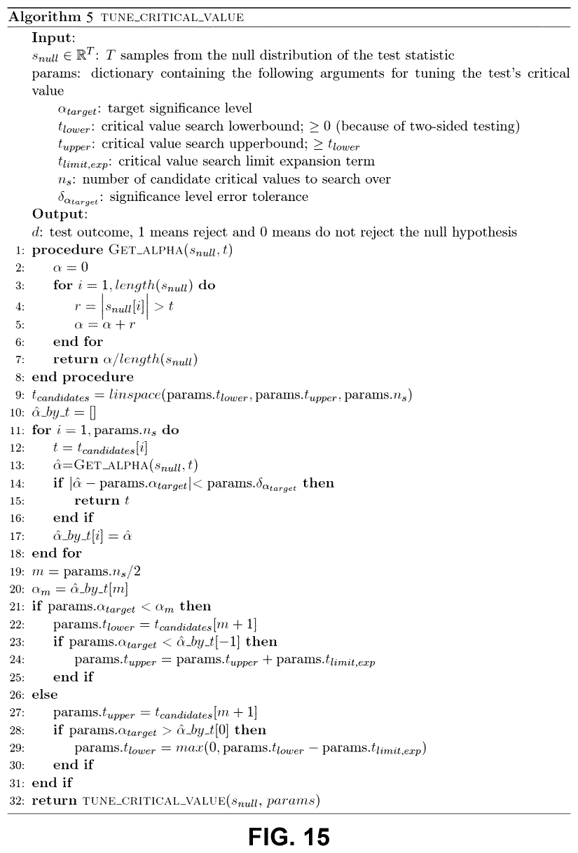

An embodiment of tune critical_value_can be seen in Algorithm 5 (FIG. 15). tune_critical_value searches over critical values to find the one yielding significance level params. α_{target}. First, it sweeps over a range of candidate critical values t_{candidates}, defined by params.nₛ, params.t_{lower}, and params.tᵤₚₚₑᵣ, and checks if any value obtains the desired significance level params.α_{target} (lines 9-18). For each t, it estimates the resulting significance level \hat{α} by calling Get_alpha (line 13), which calculates a by evaluating the two-sided test with the candidate critical value t. Get_alpha (lines 1-8) runs the test on each sample from the null distribution sₙᵤₗₗ (line 4) and calculates the fraction of samples on which the test returns a reject (line 7). If the critical value yields \hat{α} close to params.α_{target} (i.e., based on the error tolerance params.δ_{α}_{_{target}}); tune_critical_value returns the critical value (lines 14-16); otherwise, it stores the corresponding significance level and resumes the sweep (line 17).

If none of the candidate values yield significance levels close to params.α_{target}, tune_critical_value updates its search range in a fashion similar to binary search. First, it identifies the significance level of the middle candidate critical value αₘ (lines 19-20). If params.α_{target} is less than αₘ, tune_critical_value updates its search range to the right half of the original critical value search space since increasing the critical value would decrease the significance level (lines 21-22). In addition, if params.α_{target} is less than the \hat{α} associated with params.tᵤₚₚₑᵣ, params.tᵤₚₚₑᵣ needs to be sufficiently increased (i.e., by params.t_{limit,exp}) to ensure the updated search range contains a critical value yielding α_{target} (lines 23-24). Analogously, if params.α_{target} is greater than αₘ, tune_critical_value updates its search range to the left half of the original critical value search space since decreasing the critical value would increase the significance level (lines 26-27). In addition, if params.α_{target} is greater than \hat{α} associated with params.t_{lower}, params.t_{lower} needs to be sufficiently decreased (i.e., by params.t_{limit,exp} but lower bounded by 0 because of two-sided testing) to ensure the updated search range contains a critical value yielding α_{target} (lines 28-29). After updating the search range, tune critical value continues searching by recursing with the updated search parameters and returning the identified critical value (line 32).

run_hypothesis_test then calls run_test to perform the test (e.g., the two-sided test) with the tuned critical value t and the ITEs over the control and treatment groups Y, and returns the test decision d.

### EXAMPLES

For evaluation, the number of trials was set to 100 since this was sufficient for powers and significance levels to stabilize. For implementation ease, results were pre-computed across arm sizes sampled between the pilot study size to the maximum arm size and then projected interim and final sample sizes to this set. Sampling was done in increments of 25 since this was sufficient for consecutively sampled arm sizes to have comparable means and variances.

As part of the example, the disclosed framework is evaluated on a real-world clinical Phase-3 parallel-group RCT and demonstrate it for a two-arm superiority trial, a design typically adopted in clinical RCTs. The dataset for a sample trial, e.g., CHAMP (NCT01581281), was obtained from the National Institute of Neurologic Disease and Stroke (NINDS). The CHAMP study conducted an RCT to compare the effect of different medications (amitriptyline and topiramate) on mitigating headaches.

A dataset was constructed corresponding to a two-arm superiority trial. Specifically, the control arm was set to be the group exposed to amitriptyline and the treatment arm to be the group exposed to topiramate and define the ATE to be the difference between their average outcomes, where the outcome is the change in the score on the Pediatric Migraine Disability Assessment Scale between the 24-week endpoint and the baseline visit. This setup yields a dataset of 204 subjects, with 106 in the amitriptyline group and 98 in the topiramate group, where the ATE is-3.17 units, somewhat comparable to the −4.3 units reported in the study.

TAD-SIE is compared against two baseline algorithms. Both algorithms implement parallel-group RCTs following a two-arm superiority setup and therefore use the two-sample t-test for independent samples with unequal variances for hypothesis testing. The approaches differ in how they determine the final sample size.

The Fixed Sample Design baseline is a standard approach for study planning that calculates the sample size required for target power and target significance level using Eq. (1), where the ATE δ and variances,

$${σ}_{ctrl}^{2} and {σ}_{treat}^{2} ,$$

are pre-specified or estimated from a prior study. Since domain knowledge may not be available to appropriately pre-specify these parameters and since prior comparable studies do not exist for new interventions, the baseline implements a small internal pilot study to estimate these parameters. The baseline then conducts an RCT according to the calculated sample size, which is capped at a maximum arm size set by trial constraints.

Since the Fixed Sample Design baseline performs poorly in reaching a target operating point, given that it relies on noisy estimates of the ATE and variances, an adaptive design was implemented that can increase the initial sample size calculated by the Fixed Sample Design strategy in order to increase power. A TAD based on CP was adopted and specifically implement the algorithm from Chen, Y. J., et al., “Increasing the sample size when the unblinded interim result is promising,” Statistics in Medicine, vol. 23, no. 7, pp. 1023-1038, 2004 (the algorithm being incorporated by reference herein), given its simplicity, and use the sample size formula to calculate the amount by which to increase the sample size for simplicity. This baseline is referred to as Standard-TAD.

Ablations were performed on TAD-SIE in order to understand the contribution of key steps. First, the estimate moments procedure was ablated by swapping the disclosed variance estimation strategy with a naïve one to show that the disclosed approach can reach the target operating point and do so more efficiently.

Then, ablations were run on the trend-adaptive algorithm by swapping it with a standard TAD to show that an approach implementing a rule for sample size increases, based on control over significance level, will fail to reach the target operating point since increases are rare.

Finally, TAD-SIE was modified so that it performs sample size estimation based on standard hypothesis testing instead of SECRETS to show that a TAD designed for a powerful test is necessary for reaching the. target operating point.

All ablations utilize sweeping over any relevant hyperparameters to maximize the ablated algorithm's performance. The results for when target power is 80% and target significance level is 5% are reported since the effect of each ablation is independent of the target operating point.

The framework and experiments were implemented with Python using standard numerical packages and conducted experiments using 28-32 CPU cores, 2-4 GB of memory per CPU, and Intel processors (e.g., 2.4 GHz Skylake and 2.6 GHz Intel Skylake).

The hyperparameters and other inputs used by TAD-SIE and the baseline algorithms are described, then the hyperparameters used for evaluation. Finally, the computing setup used to perform experiments is described.

Hyperparameters for TAD-SIE are reported in Table 1. While most hyperparameters can be determined from prior work, step_size_scale_factor is a new hyperparameter introduced by TAD-SIE; hence, the example included sweeping over values over the domain of the hyperparameter in increments of 0.1 to characterize its effect on performance. The example further simultaneously sweeps over values for futility_power_boundary over a sufficient range in increments of 1% starting from 1% since there is no standard or recommended value; it also includes 0% since this corresponds to no futility stopping. In addition, get_outcome was set to be the function that calculates the outcome metric defined for the dataset.

The baseline algorithms use the same values used by TAD-SIE for nₚᵢₗₒₜ, α_{target}, 1−β_{target}, and nₘₐₓ, and calculate the same outcome metric. For Standard-TAD, the number of interim analyses was set to 1 since this is common in practice and perform interim analysis at the initial planned sample size by setting t=0.99 (CP is undefined at t=1) since this is ideal for assessing whether the sample size can be increased and the amount by which it needs to be increased.

TABLE 1
Hyperparameters used for TAD-SIE. The “Reference” column lists
references that support the choice of the hyperparameter value.

| Hyperparameter | Value |
|---|---|
| nₚᵢₗₒₜ | 30 |
| SI_tuning_params | {r_{train, val} = 7/3} |
| B | 100 |
| α_{target} | 5% |
| 1 − β_{target} | 80%/90% |
| nₘₐₓ | 1500 |
| step_size_scale_factor | (0, 1] |
| futility power boundary | [0, 20%] |
| Testing_params | |
| T | 100 |

For evaluation, the number of trials was set to 100 since this was sufficient for powers and significance levels to stabilize. For implementation ease, results were pre-computed across arm sizes sampled between the pilot study size to the maximum arm size and then projected interim and final sample sizes to this set. Sampling occurred in increments of 25 since this was sufficient for consecutively sampled arm sizes to have comparable means and variances.

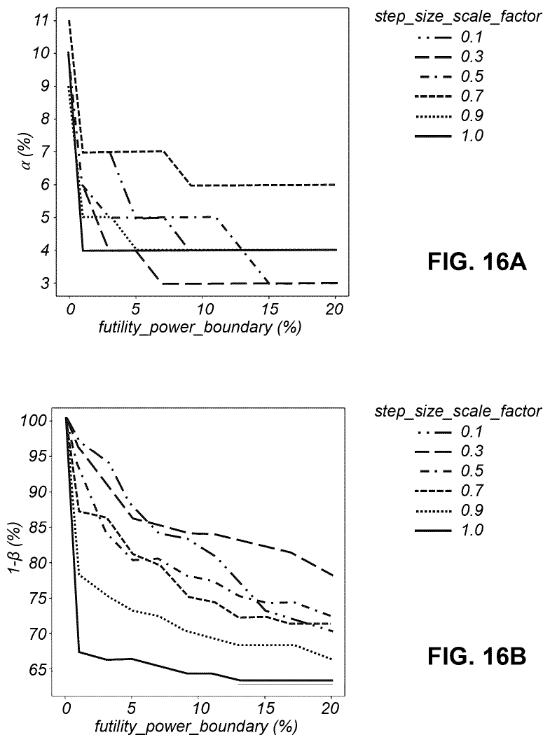

The following results demonstrate the effect of hyperparameters that govern dynamics under TAD-SIE. First, the effect of step size scale factor and futility power boundary on power and significance level are assessed to identify a range of hyperparameter values that are feasible, i.e., can reach the target operating point. Given the feasible range, the effect of step size scale factor is analyzed on the sample and time efficiencies of solutions under TAD-SIE in order to suggest hyperparameter settings for the sample-efficient and time-efficient modes. The effect of step_size_scale_factor and futility_power_boundary on TAD-SIE's performance, when target significance level is 5% and target power is 80%, is shown in FIGS. 16A and 16B. As expected, setting futility power boundary to 0% fails to control the significance level (significance levels are around 10%); increasing it reduces the significance level and power across all values for step size scale factor, although feasible solutions are less likely to exist when step size scale factor is too large, i.e., values larger than 0.7. For example, when step_size_scale_factor is 0.9, setting futility power boundary to just 1% already reduces the significance level to 5% and power to 78% and when step_size_scale_factor is 1, setting futility_power_boundary to 1% reduces the significance level to 4% and power to 67%. In contrast, when step_size_scale_factor is 0.5, setting futility power boundary between 3% and 7% obtains 5% significance level and at least 80% power, with similar results when step_size_scale_factor is 0.1 and 0.3. Similar trends hold when target power is, e.g., 90%.

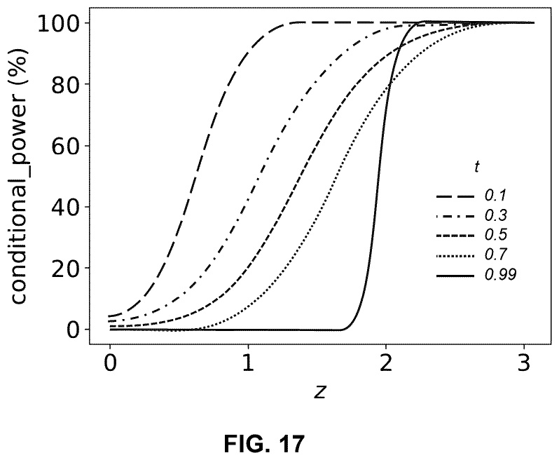

Feasible solutions are unlikely to exist when setting step_size_scale_factor to large values because doing so increases the chance for futility stopping when H₁ holds, which makes it difficult to reach target power while controlling the significance level. For example, when futility_power_boundary is 1%, 7% and 22% of trials terminate by futility when H₁ holds under a step size scale factor of 0.5 and 0.9, respectively. Setting step_size_scale_factor to a large value increases the chance for futility stopping by increasing the information fraction t, given that t is more likely to be determined by step_size_scale_factor at initial interim analyses when the current arm size is small (per line 6 in Algorithm 1). A higher information fraction shrinks CP over a large range of test statistic values, as shown in FIG. 17, thereby triggering futility stopping. Given the relationship between step_size_scale_factor and futility stopping, only solutions where step size scale factor is not too large were considered. Setting step_size_scale_factor below 0.7 works well in yielding a set of feasible solutions across typical target operating points (i.e., 80% or 90% power and 5% significance level).

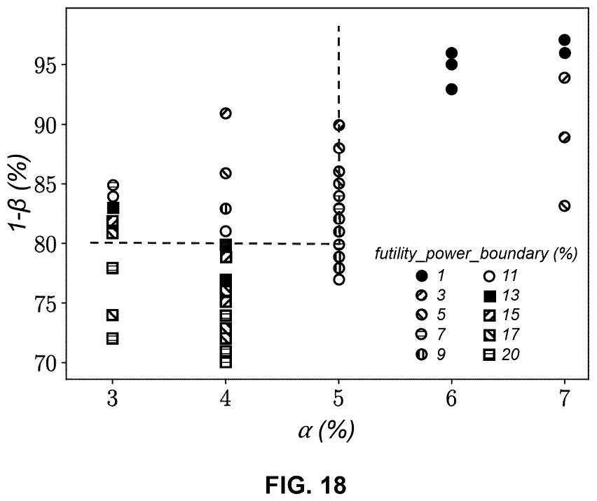

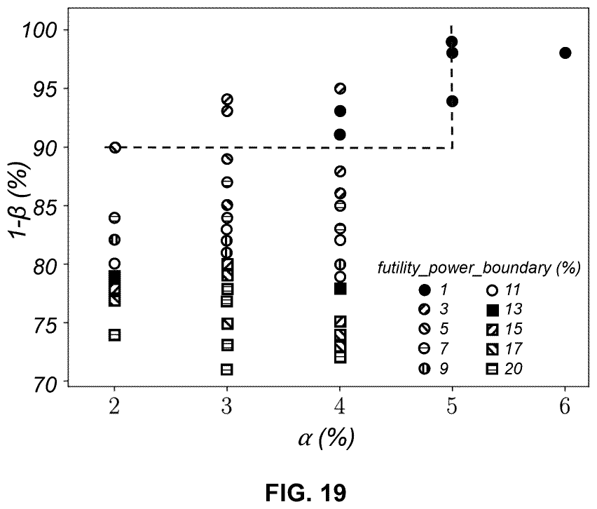

By adopting smaller values for step size scale factor, TAD-SIE is more robust to futility stopping when H₁ holds and futility stopping rates remain comparable when H₀ holds since the ATE is small. Therefore, values for futility power boundary generally transfer well, i.e., they yield operating points in a similar range across other values for step size scale factor. This phenomenon is shown in FIG. 18 when the target significance level is 5% and target power is 80%. For example, when futility_power_boundary is 1%, setting step_size_scale_factor to any value between 0.1 and 0.6 yields significance levels in a higher range (5-7%) and powers in a high range (88-97%) and when futility_power_boundary is 20%, setting step size scale factor to any value between 0.1 and 0.6 yields significance levels in a lower range (3-4%) and powers in a low range (70-78%). Among hyperparameter settings that meet the target operating point, indicated by the region enclosed by dashed lines in FIG. 18, setting futility power boundary to values near 10% work well across values for step_size_scale_factor between 0.1 and 0.6; therefore, for these examples, it is set to 11%. By applying a similar process to TAD-SIE when target power is 90% and target significance level is 5%, it is found that setting futility_power_boundary to values near 1% work well, as shown in FIG. 19 (the boundary needs to be reduced to increase power); therefore, it is set to 1% in this case.

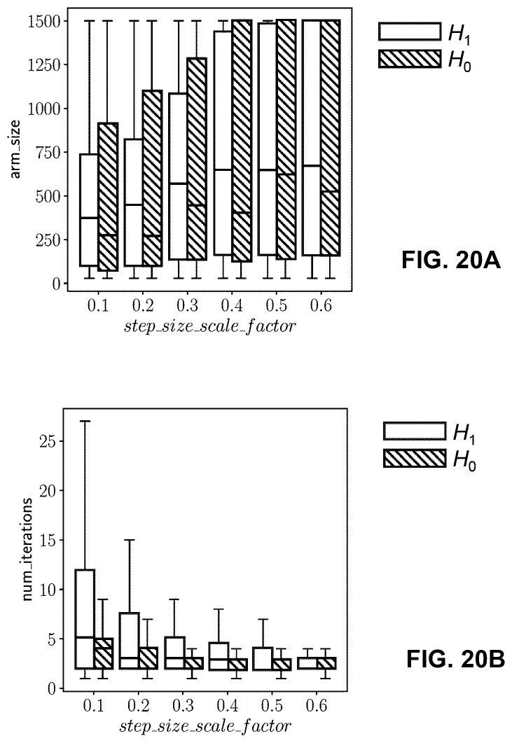

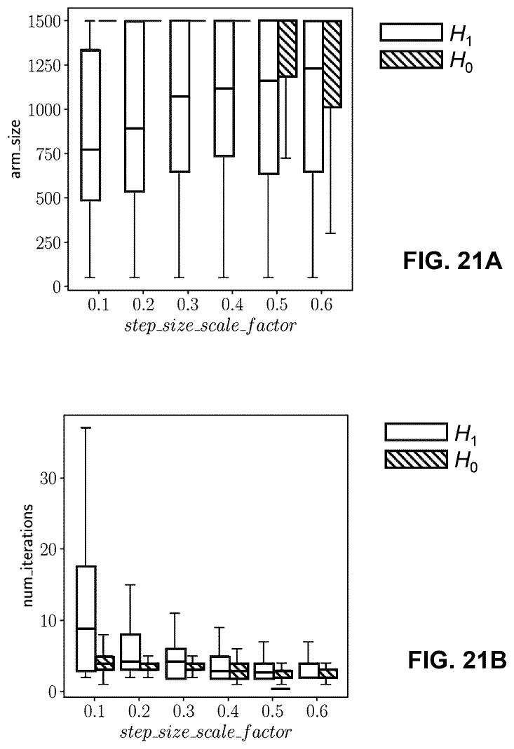

Given an effective value for futility_power_boundary, step_size_scale_factor is varied to yield solutions that reach the target operating point but are efficient in different respects, i.e., the final arm size and number of iterations incurred under TAD-SIE. This tradeoff is demonstrated when target power is 80% and target significance level is 5%, as shown in FIGS. 20A and 20B. For example, when H₁ holds and step size scale factor is 0.1, the median final arm size is 375 and the median number of iterations is 5 and when step size scale factor is 0.6, the median final arm size is 688 and the median number of iterations is 2. This tradeoff occurs because increasing step_size_scale_factor increases initial step sizes, which can overshoot and cause the algorithm to terminate earlier since the step size goes to zero in a later iteration; in addition, increasing step_size_scale_factor increases the chance for futility stopping through its effect on information fraction, which can also cause trials to terminate earlier. When H₀ holds, similar trends manifest; however, since the ATE is small, this triggers futility stopping, which generally keeps the number of iterations and arm size lower than those observed under H₁. For example, when step_size_scale_factor is 0.1, the median final arm size is 300 and the median number of iterations is 4 and when step_size_scale_factor is 0.6, the median final arm size is 538 and the median number of iterations is 2. Similar trends hold when the target power is 90% and target significance level is 5%, as shown in FIGS. 21A and 21B, although the sample size generally tops out under H₀, even when step size scale factor is low, since target power is higher. Based on these trends, step_size_scale_factor is set to 0.1 to implement a sample-efficient version of TAD-SIE, i.e., TAD-SIE-SE, and step size scale factor to 0.6 to implement a time-efficient version of TAD-SIE, i.e., TAD-SIE-TE.

TAD-SIE vs. Baselines

Given hyperparameter values for TAD-SIE, i.e., TAD-SIE-SE and TAD-SIE-TE, one can compare its performance against the baseline algorithms. TAD-SIE is able to reach desired performance under typical target operating points while the baselines cannot and obtain low accuracies. Results across the algorithms for when target power is set to 80% and target significance level is set to 5% are shown in Table 2. Fixed Sample Design results in lower power (48%) and higher significance level (9%) since it uses noisy estimates obtained from a small pilot study to estimate the required sample size. Standard-TAD improves the Fixed Sample Design by allowing the initial sample size estimated under Fixed Sample Design to be increased based on interim data; however, the actual improvement is marginal (i.e., power increases to 49% while significance level decreases to 6%) since few trials perform an increase when H₁ holds given that Standard-TAD imposes a stringent condition for when the sample size can be increased in order to control the significance level. Specifically, when H₀ holds, 7% of trials meet the criterion and 4% of trials increase the sample size, thereby preventing type-1 inflation; however, when H₁ holds, only 51% of trials meet the criterion and 17% of trials increase the sample size, thereby precluding gains in power.

TABLE 2
Performance under baseline algorithms and TAD-SIE with 1 −
β_{target} = 80% and α_{target} = 5%.

| Method | 1 − β (%) | a (%) |
|---|---|---|
| Fixed Sample Design | 48 | 9 |
| Standard-TAD | 49 | 6 |
| TAD-SIE-SE | 81 | 4 |
| TAD-SIE-TE | 80 | 4 |

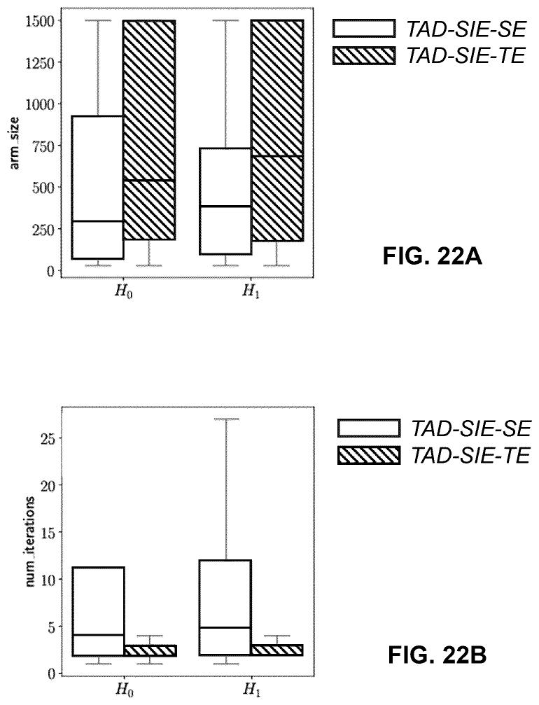

TAD-SIE improves upon Standard-TAD by integrating SECRETS to boost power and allowing trials to increase sample size without any condition while controlling the significance level with futility stopping. Specifically, both TAD-SIE-SE and TAD-SIE-TE have powers and significance levels that are comparable to the target operating point. The difference between the two solutions is that TAD-SIE-SE arrives at the target operating point with a lower sample size at the cost of more iterations while TAD-SIE-TE arrives at the target operating point in fewer iterations at the cost of higher sample size, as shown in FIGS. 22A and 22B. For example, when H₀ holds, TAD-SIE-SE has a median arm size of 300 and requires a median of 4 iterations while TAD-SIE-TE has a median arm size of 538 and requires a median of 2 iterations. Similar trends appear when H₁ holds, although the median arm sizes are higher, i.e., 375 and 688 under TAD-SIE-SE and TAD-SIE-TE, respectively, and the median number of iterations is comparable or higher, i.e., 5 and 2 under TAD-SIE-SE and TAD-SIE-TE, respectively. The increase in arm size and number of iterations under H₁ is expected since futility stopping is less likely to be invoked.

Similar trends hold when target power is 90% and target significance level is 5%, with TAD-SIE-SE and TAD-SIE-TE outperforming the baselines, though all methods show an expected increase in power, as shown in Table 3.

TABLE 3
Performance under baseline algorithms and TAD-SIE with 1 −
β_{target} = 90% and α_{target} = 5%.

| Method | 1 − β (%) | a (%) |
|---|---|---|
| Fixed Sample Design | 57 | 8 |
| Standard-TAD | 59 | 4 |
| TAD-SIE-SE | 98 | 6 |
| TAD-SIE-TE | 91 | 4 |

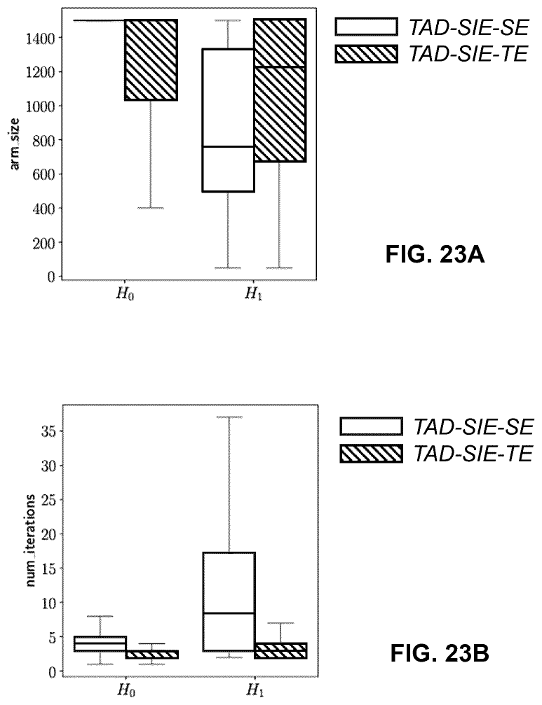

Similar tradeoffs between sample size and number of iterations under TAD-SIE-SE and TAD-SIE-TE are also observed, as shown in FIGS. 23A and 23B, though both modes show expected increases in median sample sizes and number of iterations. In addition, by aiming for a higher power, TAD-SIE-SE and TAD-SIE-TE rapidly reach the maximum arm size under H₀, hence incurring larger median arm sizes under H₀ than under H₁, which also leads to early termination, thereby keeping the number of iterations low under H₀.

### Ablation Studies

Having established TAD-SIE's effectiveness, the results from the ablation studies demonstrate the importance of each component underlying TAD-SIE. Specifically, the importance of the moment estimation procedure, trend-adaptive algorithm, and hypothesis testing algorithm are disclosed.

### Moment Estimation Strategy

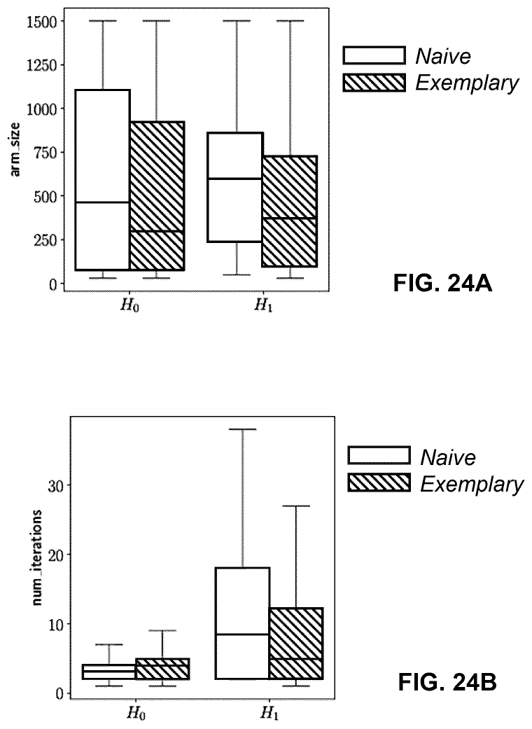

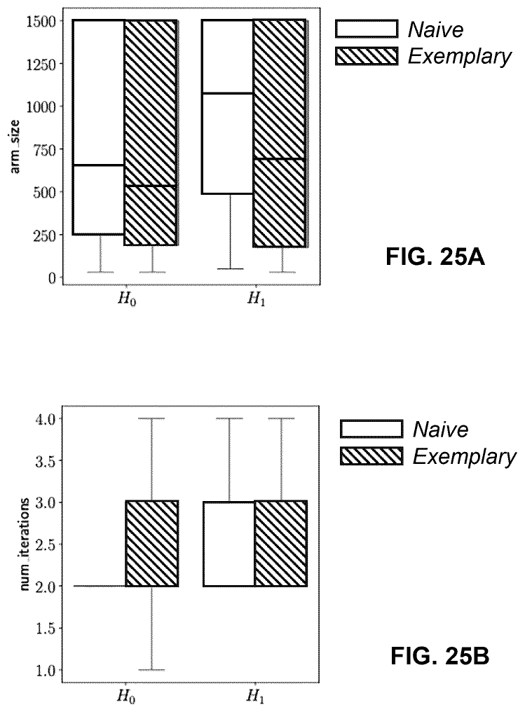

The disclosed procedure for estimating moments, specifically the variance, is necessary for reducing bias in sample size estimates under TAD-SIE. To demonstrate this, the variance calculation step in estimate moments is swapped with a naive approach that estimates variance that defines the sample size formula (Eq. (4)) by variance over the ITEs. The naive approach can either underestimate the variance, thereby lowering the final sample size at the cost of reaching target power, or overestimate the variance, thereby increasing the final sample size at the cost of lowering the algorithm's efficiency in reaching the target power. Empirically, it was found that the former result does not hold as TAD-SIE with the naive moment estimation strategy is able to reach the target operating point of 80% power and 5% significance level. Therefore, the two moment estimation strategies are compared with respect to TAD-SIE's efficiency, i.e., the final sample size and number of iterations incurred; this is shown for TAD-SIE-SE in FIGS. 24A and 24B. When H₀ holds, TAD-SIE-SE with the naive approach has a median arm size of 463 and incurs a median of 3 iterations while TAD-SIE-SE with the disclosed exemplary approach has a median arm size of 300 and incurs a median of 4 iterations. When H₁ holds, TAD-SIE-SE with the naïve approach has a median arm size of 600 and incurs a median of 8.5 iterations while TAD-SIE-SE with the exemplary approach has a median arm size of 375 and incurs a median of 5 iterations. Similar trends manifest for TAD-SIE-TE, as shown in FIGS. 25A and 25B, although the number of iterations is comparably low across both approaches due to the rapid increase in the sample size over the iterations. The results imply that the naive moment estimation strategy overestimates the variance substantially compared to the disclosed exemplary moment estimation strategy, thereby increasing the final sample size and subsequently the number of iterations required by the algorithm to reach the target operating point. The disclosed exemplary moment estimation strategy allows TAD-SIE to be more efficient.

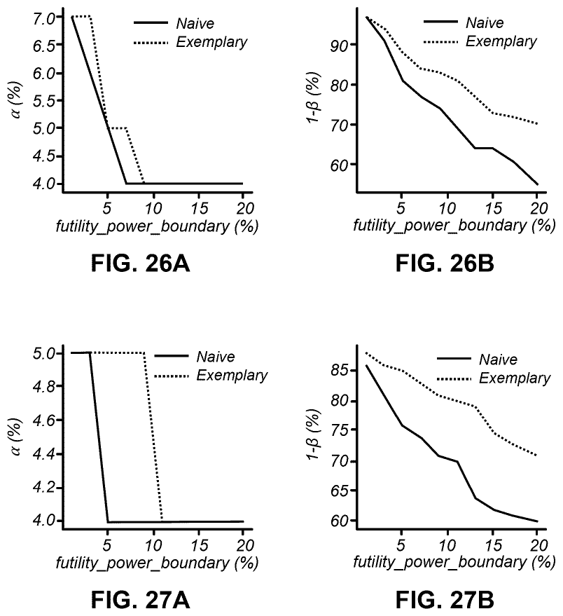

The moment estimation strategy also affects futility stopping by determining CP. To isolate its effect, one can use the disclosed moment estimation strategy for sample size calculation in get_step_size but vary the strategy used to calculate CP, i.e., the interim test statistic z in check_for_futility (line 1 of Algorithm 2). The results of this ablation for TAD-SIE-SE with target power 80% and target significance level 5% are shown in FIGS. 26A and 26B. While significance levels remain comparable under the two approaches across values of futility_power_boundary, power is lower under the ablated version of TAD-SIE that uses the naive moment estimation strategy for calculating CP. Since the naive approach overestimates variance, this shrinks the value of the interim test statistic and subsequently reduces CP (as seen in FIG. 17), thereby increasing the chance for futility stopping. The effect on futility stopping rates is negligible when H₀ holds since the magnitude of the ATE is already low and determines the futility stopping rate; e.g., when futility_power_boundary is 1%, futility stopping rates are 91% and 89% under our and the naive moment estimation strategies, respectively, and when futility_power_boundary is at least 9%, futility stopping rates are 96% under both moment estimation strategies. However, when H₁ holds, the magnitude of the ATE is not low so the deflation of the interim test statistic induced under the naive moment estimation approach causes power to drop substantially. Similar trends manifest under TAD-SIE-TE with target power 80% and target significance level 5%, as shown in FIGS. 27A and 27B. Therefore, the disclosed moment estimation strategy is essential for sustaining high power.

The disclosed trend-adaptive algorithm can be shown to be critical to TAD-SIE by swapping it with a standard trend-adaptive algorithm, which we refer to as Standard-TAD+SIE; this ablated version of the algorithm is similar to the baseline given by Standard-TAD but uses the proposed moment estimation procedure for estimating moments and uses SECRETS for hypothesis testing. As opposed to TAD-SIE, Standard-TAD+SIE yields only one solution, i.e., there is no sample-efficient or time-efficient mode since the standard trend-adaptive algorithm simply increases the sample size based on the sample size formula without introducing a hyperparameter that scales it.

Results for the ablation when the target power is 80% and target significance level is 5% are reported in Table 4.

TABLE 4
TAD-SIE (sample-efficient and time-efficient modes)
compared against an ablated version of
TAD-SIE that swaps the proposed trend-adaptive algorithm
for a standard one, with 1 − β_{target} =
80% and α_{target} = 5%. For each method,
what is reported is significance level and power, percent
of trials that increase the sample size at least once when
H₀ or H₁ holds, and percent of
trials that terminate by futility when H₀ or
H₁ holds, given by the columns, respectively.
For Standard-TAD + SIE, the entry under “% terminating by
futility” is n/a since this algorithm does not use futility stopping.

| | | % performing | % futility stopping |
|---|---|---|---|
| Method | α/(1 − β) (%) | increase (H₀/H₁) | (H₀/H₁) |
| TAD-SIE-SE | 4/81 | 71/73 | 96/19 |
| TAD-SIE-TE | 4/80 | 27/42 | 96/20 |
| Standard-TAD + SIE | 6/62 | 4/13 | n/a |

While all methods have significance levels close to 5%, TAD-SIE-SE and TAD-SIE-TE get powers close to 80% while Standard-TAD+SIE only results in 62% power. Power drops substantially under Standard-TAD+SIE because the standard trend-adaptive algorithm imposes a stringent condition based on CP that precludes trials from increasing their sample sizes after the initial sample size calculation in order to control the significance level. In particular, under Standard-TAD+SIE, only 4% of trials increase the sample size when H₀ holds, thereby controlling significance level; however, this comes at the cost of power since only 13% of trials increase the sample size when H₁ holds. In contrast, the disclosed trend-adaptive design allows trials to increase sample sizes without any condition while monitoring futility, thereby allowing TAD-SIE to reach target power and control the significance level. In particular, under TAD-SIE-SE, when H₀ holds, 71% of trials perform at least one sample size increase but 96% of trials terminate by futility; when H₁ holds, 73% of trials perform at least one sample size increase and only 19% of trials terminate by futility. Similar trends manifest under TAD-SIE-TE, although the fraction of trials that perform at least one increase declines since TAD-SIE-TE uses larger step sizes. These results establish the effectiveness of the disclosed trend-adaptive algorithm in reaching the target operating point.

SECRETS vs. Standard Hypothesis Testing

SECRETS is critical to the performance gains under TAD-SIE. To demonstrate this, TAD-SIE was appropriately modified to make it suitable for standard hypothesis testing. Specifically, estimate moments was modified to calculate the ATE and variances of the outcome over the control and treatment groups based on the original RCT data; these equations are captured in Eq. (6), where o_{control} and oₜᵣₑₐₜ are vectors that contain primary outcomes per subject in the control and treatment groups, respectively. get_step_size was also modified so that it takes in the variance for the control and treatment groups and uses the appropriate sample size formula, i.e., Eq. (1), in line 1 of Algorithm 1; it is not divided by two since Eq. (1) returns the sample size per arm. check_for_futility is similarly modified so that it takes in the two variance terms and uses the appropriate test statistic formula, i.e., Eq. (7), in line 1 of Algorithm 2. Finally, the hypothesis testing procedure from SECRETS is switched to the two-sample t-test for independent samples with unequal variances.

$$\begin{matrix}δ = Mean ( {o}_{treat} ) - Mean ( {o}_{control} ) \\ {σ}_{ctrl}^{2} = Var ( {o}_{control} ) \\ {σ}_{treat}^{2} = Var ( {o}_{treat} )\end{matrix} \qquad ( 6 )$$

$$z = \frac{δ}{\sqrt{( {σ}_{c t r l}^{2} + {σ}_{t r e a t}^{2} )} / {n}_{a}} \qquad ( 7 )$$

Results that compare TAD-SIE against the ablated version with target power of 80% and target significance level of 5% are shown in Table 5.

TABLE 5
TAD-SIE (sample-efficient and time-efficient modes)
compared against a version of TAD-SIE designed for two-sample
t-testing with 1 − β_{target} = 80% and α
_{target} = 5%. The version is indicated by the “Hypothesis
Testing Strategy” column.

| Method | Hypothesis Testing Strategy | 1 − β (%) | α (%) |
|---|---|---|---|
| TAD-SIE-SE | SECRETS | 81 | 4 |
| | Standard | 22 | 5 |
| TAD-SIE-TE | SECRETS | 80 | 4 |
| | Standard | 54 | 5 |

Specifically, for TAD-SIE-SE, swapping TAD-SIE with the version designed for standard hypothesis testing reduces power from 81% to 22% even though significance level increases from 4% to 5%; similar trends manifest for TAD-SIE-TE although the drop in power is not as large since the algorithm terminates with a larger sample size (i.e., when H₁ holds, the median final arm size is 75 under TAD-SIE-SE and 450 under TAD-SIE-TE). TAD-SIE designed for SECRETS is superior to the ablated version because SECRETS is able to boost power over standard hypothesis testing at any arm size by simulating a cross-over trial, thereby increasing the chance of reaching target power within the trial's sample size constraints.

The results show that TAD-SIE is able to reach target accuracies, given poor initial sample size estimates for a parallel-group RCT, by using a trend-adaptive algorithm designed for a powerful hypothesis testing strategy. Specifically, it leverages SECRETS for hypothesis testing, which offers substantial boosts in power at any given arm compared to standard hypothesis testing; hence, integrating this hypothesis testing algorithm into an adaptive design makes it easier to reach target powers within a trial's constraints, especially under noisy estimates of the ATE and outcome variance, as demonstrated by our ablation that adapts TAD-SIE to standard hypothesis testing. However, reaching the target operating point under SECRETS requires a new TAD since SECRETS introduces dependencies among samples, a property that violates assumptions under standard TADs.

Specifically, these ablations show that the disclosed procedure for estimating the variance properly accounts for dependencies among the samples so that sample size estimates and CP calculations for futility stopping are more accurate. In addition, since the standard TAD is ineffective at reaching target power, our TAD overcomes this limitation by removing the sample increase criterion and iteratively increasing the sample size based on improved sample size estimates, while controlling the significance level with futility stopping.

In addition, TAD-SIE can toggle between sample-efficient or time-efficient modes to realize the target operating point, thereby providing an additional degree of freedom to the designer that has not been considered before in the context of adaptive designs as two-stage adaptive designs are common. TAD-SIE implements these modes by decreasing or increasing sample size scale factor, which determines the rate at which the sample size is increased at each iteration, while adopting an adequate futility stopping threshold. Based on results from a sample RCT, default settings for these hyperparameters can be suggested.

While TAD-SIE was presented in the context of a standard parallel-group RCT, i.e., a two-arm superiority trial, it is applicable to multi-arm studies, since they reduce to pairwise comparisons between select arms, and to studies where equivalence or noninferiority is being assessed, since these only affect the test strategy used and require appropriate changes to calculations of sample size, test statistic, and CP within TAD-SIE (including SECRETS).

While we have demonstrated TAD-SIE's effectiveness on a sample RCT, TAD-SIE inherits the limitations of adaptive designs, namely that it assumes that the primary outcome is rapidly measurable, a property that does not hold for all RCTs (e.g., RCTs involving chronic conditions). If the primary outcome takes too long to measure, it may not be possible to perform an adaptation, even under the time-efficient mode of TAD-SIE, since the trial gets prolonged, making it obsolete.

On a related note, the sample-efficient mode of TAD-SIE assumes that the enrollment rate is slower than the rate at which the primary outcome is measured, a constraint that sometimes may not be feasible to satisfy since this can also prolong the trial, given that TAD-SIE-SE can require more than two iterations. If the enrollment rate is faster, however, TAD-SIE-SE cannot guarantee sample size reductions since by the time the algorithm terminates with the true final sample size, the number of recruited participants could exceed that value.

### Clinical RCT Datasets

Various phase 3 clinical trial datasets were used to assess the performance of SECRETS.

CHAMP: The CHAMP study evaluated whether different medications could reduce headache frequency and headache effects among children and adolescents suffering from migraines. To assess this, the study implemented an RCT containing a placebo group and two treatment groups receiving amitriptyline and topiramate, respectively. At the end of the trial, treatment effects were measured on various outcomes, including change in headache frequency, number of headache days, and headache-related disability scores, all relative to baseline measurements. Based on data from 328 subjects collected over 24 weeks, the study did not find any clinically significant between-group differences across the health outcomes.

For experiments testing the frameworks, the change in the Pediatric Migraine Disability Assessment (PedMIDAS) score was used as the metric defining the ATE and the amitriptyline and topiramate as the control and treatment groups, respectively, because the corresponding ATE was more statistically significant among the ATEs defined by other continuous metrics and arm pairs. To calculate this ATE, the study analyzed a subset of subjects monitored over two visits, i.e., one at baseline and another near the 24-week endpoint, which comprised 211 subjects with 107 and 104 subjects in the amitriptyline and topiramate groups, respectively. After applying the same criterion, 204 subjects were extracted from the original dataset, with 106 in the amitriptyline group and 98 in the topiramate group. From the extracted dataset, the ATE was calculated to be −3.17 units, somewhat comparable to the −4.3 units reported in the study.

ICARE: The ICARE study evaluated whether a new motor training program (Accelerated Skill Acquisition Program or ASAP) could reduce upper extremity disability among patients with motor stroke more effectively than usual customary care (UCC). To assess this, the study implemented an RCT containing a treatment group exposed to ASAP, a control group exposed to dose-equivalent usual customary care (DEUCC), and another control group exposed to UCC with no constraint on the dose. At the end of the trial, treatment effects were assessed over various outcomes, including changes in Wolf Motor Function Test (WMFT) time, Stroke Impact Scale (SIS) scores, and arm muscle torque. Based on data from 304 patients collected over one year, the study did not find any clinically significant between-group differences in a subset of these scores, i.e., WMFT and SIS hand function score.

For experiments testing the frameworks, the change in arm muscle torque based on shoulder flexors was used as the metric defining the ATE, and the DEUCC and ASAP as the control and treatment groups, respectively, to speed up experiment time since detecting this ATE with high power and low significance level required a relatively small sample size for the baseline method. After applying the study's data processing protocol to the original dataset, we extracted data from 183 subjects, with 93 in the control group and 90 in the treatment group, from visits at the baseline and one-year endpoints (the number of subjects analyzed per group is equal to that reported in the study's analysis). From the extracted dataset, the ATE was calculated to be −3.00 units, close to the −2.99 reported in the study.

MGTX: The MGTX study investigated whether thymectomy combined with standard prednisone therapy could treat Myasthenia Gravis more effectively than prednisone therapy alone. To assess this, the study implemented an RCT, in which the control arm received prednisone therapy over three years and the treatment arm underwent thymectomy and received the same prednisone therapy; at the end of the trial, treatment effects were measured with respect to the Quantitative Myasthenia Gravis (QMG) total score and required prednisone dose, both averaged over the study period. Based on data from 126 subjects collected over three years, the study found that thymectomy improved health outcomes with clinical significance; the time-weighted average QMG scores decreased by an average of 2.85 units and the time-weighted average prednisone dose also decreased by an average of 22 mg.

For experiments testing the frameworks, the time-weighted average QMG total score was used as the outcome defining the ATE, because it was easier to reproduce. The study calculated the ATE by analyzing a subset of the population monitored over the three-year window (14 patient visits or timepoints), which comprised 62 subjects and 56 subjects in the treatment and control groups, respectively. The study's data processing protocol was followed, and from the original dataset, a dataset with 49 subjects in the treatment group and 47 subjects in the control group was extracted. From the extracted dataset, the ATE was calculated to be −2.70 units, comparable to the −2.85 units reported in the study.

Across all datasets, SECRETS obtains better power and comparable significance level compared to Standard, and the gains in power translate into reductions in the number of subjects required to obtain a desired power 1−β_{target} and significance level α_{target}. SECRETS is able to increase power by consistently reducing the variance in the ATE estimate, albeit at the cost of higher error in the ATE estimate under the alternative setting. For example, on the CHAMP dataset (Table 6), for an arm size (nₐ) of 550, Standard has a power (1−β) of 53% and a significance level (α) of 5% while SECRETS has a power of 78% and a significance level of 5%. By increasing the power at a given arm size, SECRETS is able to converge close to the desired statistical operating point of 80% power (1−β_{target}) and 5% significance level (α_{target}) at an arm size of 550 while Standard does so at an arm size of 1130. Hence, SECRETS reduces the number of required samples by almost 51% or 580 subjects per arm.

TABLE 6
Standard vs. SECRETS on the CHAMP Dataset

| nₐ | Method | 1 − β (%) | α (%) | 1 − \hat{β} (%) | shift factor |
|---|---|---|---|---|---|
| 550 | Standard | 53 | 5 | 52 | 2.01 |
| | SECRETS | 78 | 5 | 73 | 2.58 |
| 1130 | Standard | 81 | 4 | 84 | 2.93 |
| | SECRETS | 97 | 6 | 95 | 3.62 |

TABLE 7
Standard vs. SECRETS on the ICARE Dataset

| nₐ | Method | 1 − β (%) | α (%) | 1 − \hat{β} (%) | shift factor |
|---|---|---|---|---|---|
| 1250 | Standard | 27 | 4 | 30 | 1.43 |
| | SECRETS | 81 | 6 | 81 | 2.83 |
| 5207 | Standard | 81 | 6 | 81 | 2.83 |
| | SECRETS | 100 | 6 | 100 | 5.56 |

TABLE 8
Standard vs. SECRETS on the MGTX Dataset

| nₐ | Method | 1 − β (%) | α (%) | 1 − \hat{β} (%) | shift factor |
|---|---|---|---|---|---|
| 30 | Standard | 70 | 5 | 63 | 2.30 |
| | SECRETS | 79 | 6 | 69 | 2.45 |
| 40 | Standard | 81 | 5 | 75 | 2.63 |
| | SECRETS | 87 | 5 | 80 | 2.80 |

To assess the factors contributing to the increased power under SECRETS, the distributions of the ATE estimates are analyzed using a power estimation model (Eq. (8)).

$$1 - \hat{β} = Φ [ - {z}_{1 - \frac{{α}_{t a r g e t}}{2}} + \frac{\vert {\hat{μ}}_{o} - {\hat{μ}}_{1} \vert}{\hat{σ}} ] \qquad ( 8 )$$

The equation states that if the underlying distribution of the sample statistic is normal, then the power of the two-sided z-test, 1−\hat{β}, is approximately given by the cumulative distribution function Φ, evaluated at the critical value

$${z}_{1 - \frac{{α}_{t a r g e t}}{2}} ,$$

i.e., the

$$( 1 - \frac{{α}_{t a r g e t}}{2} ) \%$$

of a standard normal distribution, shifted by a constant, which we refer to as the shift factor. The shift factor is given by Eq. (9), where parameters \hat{μ}ₒ and \hat{μ}₁ correspond to the means of the distribution of the sample statistic under the null and alternative settings, respectively, and parameter \hat{σ} is the standard deviation of the distributions.

$$shift factor = \frac{\vert {\hat{μ}}_{o} - {\hat{μ}}_{1} \vert}{\hat{σ}} \qquad ( 9 )$$

First, it is assessed whether the model is suitable by comparing its power estimates against the measured powers. For example, under an arm size of 550, the model estimates a power (1−β) of 52% and 73% compared to the measured powers (1−β) of 53% and 78% for Standard and SECRETS, respectively. The model estimates the measured powers somewhat well; therefore, one can analyze the shift factor to assess how differences in the distribution of the ATE estimates explain differences in power between Standard and SECRETS. First, it is noted that the means are further separated under Standard, i.e., the distance between the means (|\hat{μ}₁−\hat{μ}₀|) is 3.27 and 1.85 under Standard and SECRETS, respectively. Compared to SECRETS, Standard better separates the means because it has lower error in the ATE estimate under the alternative setting (\hat{μ}₁−μ_{1,target}) while both have comparably low error in the ATE estimate under the null setting (μ₀−μ₀). Despite their enhanced mean separation under Standard, the distributions have lower variance under SECRETS than under Standard, i.e., the average standard deviation (\hat{σ}) is 1.63 and 0.72 under Standard and SECRETS, respectively. The lower variance under SECRETS outweighs the advantage of better mean separation under Standard, giving SECRETS a higher shift factor of 2.58 compared to 2.01 under Standard, which translates into higher power. Similar trends hold for an arm size of 1130, as well as on the ICARE (Table 7) and MGTX datasets (Table 8).

Various modifications may be made to the systems, methods, apparatus, mechanisms, techniques, and portions thereof described herein with respect to the various figures, such modifications being contemplated as being within the scope of the invention. For example, while a specific order of steps or arrangement of functional elements is presented in the various embodiments described herein, various other orders/arrangements of steps or functional elements may be utilized within the context of the various embodiments. Further, while modifications to embodiments may be discussed individually, various embodiments may use multiple modifications contemporaneously or in sequence, compound modifications and the like.

Although various embodiments which incorporate the teachings of the present invention have been shown and described in detail herein, those skilled in the art can readily devise many other varied embodiments that still incorporate these teachings. Thus, while the foregoing is directed to various embodiments of the present invention, other and further embodiments of the invention may be devised without departing from the basic scope thereof. As such, the appropriate scope of the invention is to be determined according to the claims.

What is claimed is:

1. A method for making a randomized control trial (RCT) more data efficient, comprising:
receiving data of a plurality of subjects, the data including control data and treatment data, each subject of the plurality of subjects being in one arm of a plurality of arms of an already-conducted RCT, the plurality of arms including a first arm and a second arm, the data being free of data from a cross-over trial and external data;
estimating a plurality of individual treatment effects (ITEs) by utilizing synthetic intervention (SI) to estimate a counterfactual treatment response for each patient in the first arm and a counterfactual control response for each patient in the second arm, where the plurality of ITEs include an ITE for each patient in the first arm and the second arm; and
conducting a two-sided hypothesis test, using the ITE for each patient and the control data, by:
estimating a null distribution of the ITE for each patient under a null hypothesis by repeatedly resampling the control data to provide samples, and running SI with the samples;
using the null distribution to determine a critical value achieving a desired significance level; and
conducting the two-sided hypothesis test using the critical value.

2. The method of claim 1, wherein the first arm is a control arm, and the second arm is a corresponding treatment arm.

3. The method of claim 2, wherein the plurality of arms consists of the first arm and the second arm.

4. The method of claim 1, further comprising reporting results of the two-sided hypothesis test, wherein the plurality of subjects includes fewer subjects than a standard RCT would require to reach a target power and significance level with respect to a target average treatment effect (ATE).

5. The method of claim 1, further comprising, prior to using SI to estimate the counterfactual treatment response, tuning at least one hyperparameter of the SI, by splitting the control data into training and validation sets, initializing a set of best hyperparameters and sweeping over a range of candidate values for ridge regression regularization strength (λᵣᵣ) and singular value truncation threshold (λₛ).

6. The method of claim 5, further comprising tracking validation performance under each hyperparameter configuration, where the validation performance is a R² score between true post-intervention validation data and corresponding SI-derived predictions.

7. The method of claim 6, wherein, to obtain SI-derived predictions per unit, SI is run with candidate hyperparameters, training data as donor data, and a target unit data, and wherein the SI is configured to estimate a post-intervention trajectory for a target unit by denoising donor data, and using donor weights to predict a target unit's post-intervention trajectory from the donor data, where the donor weights are learned with ridge regression on pre-intervention and/or baseline data.

8. The method of claim 7, further comprising updating the set of best hyperparameters if the validation performance improves.

9. The method of claim 1, further comprising conducting a pilot study to provide initial estimates on parameters defining a standard sample size formula for a one-sample two-sided test.

10. The method of claim 9, wherein the conducting the pilot study includes estimating a variance of an average treatment effect, and estimating a variance of a set of hypothetical individual treatment effects based on the estimated variance of an average treatment effect.

11. The method of claim 9, further comprising implementing a trend-adaptive algorithm configured to iteratively accrue RCT data, initialized from the pilot study.

12. The method of claim 11, wherein the trend-adaptive algorithm includes iteratively refining estimates to converge to an accurate estimate of a target sample size needed for a target power while stopping for futility to control a significance level.

13. The method of claim 11, further comprising terminating the trend-adaptive algorithm if it is determined to be futile.

14. A non-transitory computer-readable storage device containing instructions that, when executed by a processor, cause the processor to perform the method of claim 1.

15. A system, comprising:
one or more processing units;
a memory coupled to the one or more processing units; and
a non-transitory computer-readable storage device operably coupled to the one or more processing units, the non-transitory computer-readable storage device containing instructions that, when executed by the one or more processing units, cause the one or more processing units to, collectively, perform the method of claim 1.
